SEQUENCE LISTING (20 sequences)

SEQ ID NO: 1 - DNA, 27 bp, Artificial Sequence
  Description of Artificial Sequence Synthetic oligonucleotide primer
ctctgttctccagggaccaagtcagat

SEQ ID NO: 2 - DNA, 24 bp, Artificial Sequence
  Description of Artificial Sequence Synthetic oligonucleotide primer
cctacactctggggatgcggagat

SEQ ID NO: 3 - DNA, 37 bp, Artificial Sequence
  Description of Artificial Sequence Synthetic oligonucleotide probe
gactgactgactgactgactgaccccactgcctagaa

SEQ ID NO: 4 - DNA, 26 bp, Artificial Sequence
  Description of Artificial Sequence Synthetic oligonucleotide primer
gagggagaagagaggtgtgagaggga

SEQ ID NO: 5 - DNA, 24 bp, Artificial Sequence
  Description of Artificial Sequence Synthetic oligonucleotide primer
catctgggctctctgctgcttgag

SEQ ID NO: 6 - DNA, 42 bp, Artificial Sequence
  Description of Artificial Sequence Synthetic oligonucleotide probe
gactgactgactgactgactgactgactgtgcccccggagag

SEQ ID NO: 7 - DNA, 22 bp, Artificial Sequence
  Description of Artificial Sequence Synthetic oligonucleotide primer
ttggccttgggagacttctggg

SEQ ID NO: 8 - DNA, 24 bp, Artificial Sequence
  Description of Artificial Sequence Synthetic oligonucleotide primer
ttgtcacacacactgcctgggaat

SEQ ID NO: 9 - DNA, 47 bp, Artificial Sequence
  Description of Artificial Sequence Synthetic oligonucleotide probe
gactgactgactgactgactgactgactgactgcaggaggtggctca

SEQ ID NO: 10 - DNA, 21 bp, Artificial Sequence
  Description of Artificial Sequence Synthetic oligonucleotide primer
aagtgttgcccctgcaggaat

SEQ ID NO: 11 - DNA, 21 bp, Artificial Sequence
  Description of Artificial Sequence Synthetic oligonucleotide primer
gagtggaactgctgggccata

SEQ ID NO: 12 - DNA, 52 bp, Artificial Sequence
  Description of Artificial Sequence Synthetic oligonucleotide probe
gactgactgactgactgactgactgactgactgacttgtggggctccctaga

SEQ ID NO: 13 - DNA, 20 bp, Artificial Sequence
  Description of Artificial Sequence Synthetic oligonucleotide primer
ctcaccggcacgtaatgcac

SEQ ID NO: 14 - DNA, 20 bp, Artificial Sequence
  Description of Artificial Sequence Synthetic oligonucleotide primer
ccctccagaccaccatctcg

SEQ ID NO: 15 - DNA, 57 bp, Artificial Sequence
  Description of Artificial Sequence Synthetic oligonucleotide probe
gactgactgactgactgactgactgactgactgactgaccttggttgaggagacagc

SEQ ID NO: 16 - DNA, 22 bp, Artificial Sequence
  Description of Artificial Sequence Synthetic oligonucleotide primer
aagcagctggctctacaaagca

SEQ ID NO: 17 - DNA, 19 bp, Artificial Sequence
  Description of Artificial Sequence Synthetic oligonucleotide primer
agcatcctttggggcttca

SEQ ID NO: 18 - DNA, 63 bp, Artificial Sequence
  Description of Artificial Sequence Synthetic oligonucleotide probe
gactgactgactgactgactgactgactgactgactgactgactgactgcctgctgggagcct

SEQ ID NO: 19 - DNA, 59 bp, Homo sapiens
  CDS (15)..(59)
cttgaggagcgaggatggcagtcaacaaaggcctcaccttgctggatggagacctccct

SEQ ID NO: 20 - protein, 15 aa, Homo sapiens
MAVNKGLTLLDGDLP

(19) United States
(12) Patent Application Publication
(10) Pub. No.: US 2007/0248959 A1
(43) Pub. Date: Oct. 25, 2007

(54) Genetic markers for obesity

(75) Inventors: Jose M. Ordovas, Framingham, MA (US); Lu Qi, Chestnut Hill, MA (US); Andrew Greenberg, Newton, MA (US); Dolores Corella, Valencia (ES)

Correspondence Address: DAVID S. RESNICK, 100 SUMMER STREET, NIXON PEABODY LLP, BOSTON, MA, 02110-2131, US

(73) Assignee: TUFTS UNIVERSITY, Medford, MA (US)

(21) Appl. No.: 11/384,619

(22) Filed: Mar. 20, 2006

Related U.S. Application Data
(63) Continuation of application No. PCT/US04/18743, filed on Jun. 10, 2004.
(60) Provisional application No. 60/504,830, filed on Sep. 22, 2003.
(60) Provisional application No. 60/519,109, filed on Nov. 12, 2003.
(60) Provisional application No. 60/544,524, filed on Feb. 13, 2004.

(51) Int. Cl. C12Q 1/68

(52) U.S. Cl. 435/6

(57) ABSTRACT

The present invention is directed to new genetic variants or polymorphisms at the perilipin locus (PLIN) including PLIN1: 6209T (allele 1)>C (allele 2); PLIN3 10171 (allele 1) A>T (allele 2); PLIN4: 11482G (allele 1)>A (allele 2); PLIN5: 13041A (allele 1)>G (allele 2) and PLIN6: 14995A (allele 1)>T (allele 2), and their use in diagnostic and prognostic applications for obesity and obesity-related diseases, such as metabolic syndrome and cardiovascular disease.

### CROSS REFERENCE

This application claims the benefit of U.S. Provisional Application No. 60/504,830 filed on Sep. 22, 2003, U.S. Provisional Application No. 60/519,109 filed on Nov. 12, 2003 and U.S. Provisional Application No. 60/544,524 filed on Feb. 13, 2004.

### GOVERNMENT SUPPORT

This invention was supported by NIH/NHLBI grant no. HL54776 and contracts 53-K06-5-10 and 58-1950-9-001 from the U.S. Department of Agriculture. The Government of the United States has certain rights thereto.

### BACKGROUND

During the evolution, the human body has developed ingenious ways to cope with lack of calorie intake, and only recently have we began to realize the complexity of these metabolic networks. During the present times of abundance in calorie input in the developed world, this intricate and complex system has began to work against us resulting in severe epidemic of obesity and related metabolic diseases.

Adipose tissue is an essential component in human body. However, too much body fat results in obesity, a serious medical condition that currently affects about a third of adults in the United States, and about 14% of children and adolescents. The abundance of energy sources and the sedentary lifestyle in developed countries has made obesity a world-wide phenomenon. In the United States, obesity can currently be said to be the second leading cause of preventable death after smoking (www.obesity.org).

Obesity is a typical multifactorial disease caused by a combination of environmental and genetic factors. Strong evidence for a genetic component to human obesity can be seen, e.g., in the familial clustering and the high concordance of body composition in monozygotic twins. However, the role of genetic factors is complex and probably determined by interaction of several genes, each of which may have relatively small effects. Such genes are called “susceptibility” genes and their phenotypic effects are seen in combination with each other as well as with environmental factors such as nutrient intake, physical activity, and smoking.

To date, at least about 80 genes have been reported to be associated with obesity (see, e.g., Obesity Gene Map Database at http://obesitygene.pbrc.edu). Many of these genes play a role in the regulation of formation and maintenance of adipose tissue.

Obesity is often associated with other diseases. For example, a “metabolic cluster” associated with abdominal obesity and including glucose intolerance, dyslipidemia, and high blood pressure, also sometimes called the metabolic syndrome X (Reaven, 1988) or the abdominal obesity-metabolic syndrome (Bjorntorp, 1991). Fundamental to this symptomatic association appears to be the close interaction of abdominal fat patterning, total body adiposity, and insulin resistance. Obesity is also often a pre-existing condition to adult onset non-insulin dependent diabetes mellitus (Type II diabetes) and a myriad of other diseases. Despite of advances in the knowledge of adipose tissue metabolism, current regimes treating disorders of adipose tissue metabolism are still inadequate and development of novel therapies would be desirable.

### SUMMARY OF INVENTION

The present invention is directed to new genetic variants or polymorphisms at the perilipin locus and their use in diagnostic and prognostic applications for obesity and related metabolic diseases.

The invention provides for a method of determining an increased risk of obesity and obesity-related diseases in an individual comprising the steps of: a) genotyping the PLIN1 6209T/C, PLIN3 10171A/T, PLIN4 11482G/A, PLIN5 13041A/G, PLIN6 14995 A/T loci from a biological sample taken from the individual; b) creating a haplotype based on the PLIN genotypes as determined in step (a); and c) correlating the haplotype with the ethnic background of the individual, wherein a haplotype selected from the group of consisting of PLIN5-G/PLIN6T; PLIN5-A/PLIN6-T; PLIN1-T/PLIN4-G/PLIN5-G/PLIN6-T; PLIN1-T/PLIN4-G; PLIN1-T/PLIN4-G/PLIN5-A/PLIN6-A; PLIN1-T/PLIN3-A/PLIN4-APLIN5-A/PLIN6-T; PLIN1-T/PLIN3-A/PLIN/4-A/PLIN5-G/PLIN6-T; PLIN4-A/PLIN5-A/PLIN6-T; PLIN4-A/PLIN5-G/PLIN6-T; PLIN4-G/PLIN5-G/PLIN6-A; PLIN1-T/PLIN3-A; correlated to the ethnic background of the individual is indicative of increased risk of obesity and obesity-related diseases in the individual.

In one embodiment, a method of determining an increased risk of obesity and obesity-related diseases in an individual of Caucasian descent is provided comprising the steps of: a) genotyping the PLIN1 6209T/C, PLIN4 11482G/A, PLIN5 13041A/G, PLIN6 14995 A/T loci from a biological sample taken from the individual; b) creating a haplotype based on the PLIN genotypes as determined in step (a); and c) correlating the haplotype with the ethnic background of the individual, wherein a haplotype selected from the group of consisting of PLIN5-G/PLIN6T; PLIN5-A/PLIN6-T; and PLIN1-T/PLIN4-G/PLIN5-G/PLIN6-T is indicative of increased risk of obesity and obesity-related diseases in the individual of Caucasian descent.

In one embodiment, a method of determining an increased risk of obesity and obesity-related diseases in an individual of Mediterranean descent is provided comprising the steps of: a)genotyping the PLIN1 6209T/C, PLIN4 11482G/A, PLIN5 13041A/G, PLIN6 14995 A/T loci from a biological sample taken from the individual; b) creating a haplotype based on the PLIN genotypes as determined in step (a); and c) correlating the haplotype with the ethnic background of the individual, wherein a haplotype selected from the group of consisting of PLIN1-T/PLIN4-G; PLIN1-T/PLIN4-G/PLIN5-A/PLIN6-A; PLIN1-T/PLIN4-G/PLIN5-G/PLIN6-T is indicative of increased risk of obesity and obesity-related diseases in the individual of Mediterranean descent.

In one embodiment, a method of determining an increased risk of obesity and obesity-related diseases in an individual of Malayan descent is provided comprising the steps of: a) genotyping the PLIN1 6209T/C, PLIN3 10171A/T, PLIN4 11482G/A, PLIN5 13041A/G, PLIN6 14995 A/T loci from a biological sample taken from the individual; b) creating a haplotype based on the PLIN genotypes as determined in step (a); and c) correlating the haplotype with the ethnic background of the individual, wherein a haplotype selected from the group of consisting of PLIN1-T/PLIN3-A/PLIN4-A/PLIN5-A/PLIN6-T; PLIN1-T/PLIN3-A/PLIN/4-A/PLIN5-G/PLIN6-T; PLIN4-A/PLIN5-A/PLIN6-T; PLIN4-A/PLIN5-G/PLIN6-T; PLIN4-G/PLIN5-G/PLIN6-A; PLIN1-T/PLIN3-A is indicative of increased risk of obesity and obesity-related diseases in the individual of Malayan descent.

In one embodiment, a method of determining an increased risk of obesity and obesity-related diseases in an individual of Indian descent is provided comprising the steps of: a) genotyping the PLIN1 6209T/C, PLIN3 10171A/T, PLIN4 11482G/A, PLIN5 13041A/G, PLIN6 14995 A/T loci from a biological sample taken from the individual; b) creating a haplotype based on the PLIN genotypes as determined in step (a); and c) correlating the haplotype with the ethnic background of the individual, wherein a haplotype selected from the group of consisting of PLIN1-T/PLIN3-A/PLIN4-A/PLIN5-A/PLIN6-T; PLIN4-A/PLIN5-A/PLIN6-T; PLIN4-G/PLIN5-G/PLIN6-T; and PLIN1-T/PLIN3-A is indicative of increased risk of obesity and obesity-related diseases in the individual of Indian descent.

In one embodiment, a method of determining an increased risk of obesity and obesity-related diseases in an individual of Caucasian descent is provided comprising genotyping the PLIN5 13041A/G and PLIN6 14995 A/T loci from the biological sample taken from the individual, wherein homozygosity of allele G in the PLIN5 locus or homozygosity of allele T in the PLIN 6 locus is indicative of increased risk of obesity and obesity-related diseases in the individual of Caucasian descent.

In one embodiment, a method of determining an increased risk of obesity and obesity-related diseases in an individual of Malayan or Indian descent is provided comprising genotyping the PLIN6 14995 A/T loci from the biological sample taken from the individual, wherein homozygosity of allele T in the PLIN 6 locus is indicative of increased risk of obesity and obesity-related diseases in the individual of Malayan or Indian descent.

In one embodiment, a method of determining an increased risk of obesity and obesity-related diseases in an individual of Malayan or Indian descent is provided comprising genotyping the PLIN4 11482 G/A loci from the biological sample taken from the individual, wherein homozygosity of allele A in the PLIN4 locus is indicative of increased risk of obesity and obesity-related diseases in the individual of Malayan or Indian descent.

In one embodiment, a method of determining an increased risk of obesity and obesity-related diseases in an individual of Malayan or Indian descent is provided comprising genotyping the PLIN5 13041 A/G loci from the biological sample taken from the individual, wherein homozygosity of allele G in the PLIN 5 locus is indicative of increased risk of obesity and obesity-related diseases in the individual of Malayan or Indian descent.

In one embodiment, the individual whom an increased risk of obesity and obesity-related diseases is assessed is a woman.

In one embodiment, the individual whom an increased risk of obesity and obesity-related diseases is assessed has been subject to weight reducing diet.

In one embodiment, the obesity-related disease is cardiovascular disease.

In one embodiment, the obesity related disease is metabolic syndrome.

In another embodiment, a method of determining a decreased risk of obesity and obesity-related diseases in an individual is provided comprising the steps of: a) genotyping the PLIN1 6209T/C, PLIN3 10171A/T, PLIN4 11482G/A, PLIN5 13041A/G, PLIN6 14995 A/T loci from a biological sample taken from the individual; b) creating a haplotype based on the PLIN genotypes as determined in step (a); and c) correlating the haplotype with the ethnic background of the individual, wherein a haplotype selected from the group of consisting of PLIN5-A/PLIN6-A; PLIN1-C/PLIN4-G/PLIN5-A/PLIN6-A; PLIN1-C/PLIN4-A; PLIN1-C/PLIN4-A/PLIN5-A/PLIN6-A; PLIN1-T/PLIN3-T/PLIN4-G/PLIN5-A/PLIN6-A; PLIN1-C/PLIN3-A/PLIN/4-G/PLIN5-A/PLIN6-A; and PLIN1-C/PLIN3-T correlated to the ethnic background of the individual is indicative of decreased risk of obesity and obesity-related diseases.

In one embodiment, a method of determining a decreased risk of obesity and obesity-related diseases in an individual of Caucasian descent is provided comprising the steps of: a) genotyping the PLIN1 6209T/C, PLIN4 11482G/A, PLIN4 13041A/G, PLIN6 14995 A/T loci from a biological sample taken from the individual; b) creating a haplotype based on the PLIN genotypes as determined in step (a); and c) correlating the haplotype with the ethnic background of the individual, wherein a haplotype selected from the group of consisting PLIN5-A/PLIN6-A and PLIN1-C/PLIN4-G/PLIN5-A/PLIN6-A is indicative of decreased risk of obesity and obesity-related diseases in the individual of Caucasian descent.

In one embodiment, a method of determining a decreased risk of obesity and obesity-related diseases in an individual of Mediterranean descent is provided comprising the steps of: a) genotyping the PLIN1 6209T/C, PLIN4 11482G/A, PLIN5 13041A/G, PLIN6 14995 A/T loci from a biological sample taken from the individual; b) creating a haplotype based on the PLIN genotypes as determined in step (a); and c) correlating the haplotype with the ethnic background of the individual, wherein a haplotype selected from the group of consisting of PLIN1-C/PLIN4-A and PLIN1-C/PLIN4-A/PLIN5-A/PLIN6-A is indicative of decreased risk of obesity and obesity-related diseases in the individual of Mediterranean descent.

In one embodiment, a method of determining a decreased risk of obesity and obesity-related diseases in an individual of Malayan descent is provided comprising the steps of: a) genotyping the PLIN1 6209T/C, PLIN3 10171A/T, PLIN4 11482G/A, PLIN5 13041A/G, PLIN6 14995 A/T loci from a biological sample taken from the individual; b) creating a haplotype based on the PLIN genotypes as determined in step (a); and c) correlating the haplotype with the ethnic background of the individual, wherein a haplotype selected from the group of consisting of PLIN1-T/PLIN3-T/PLIN4-G/PLIN5-A/PLIN6-A; PLIN1-C/PLIN3-A/PLIN4-G/PLIN5-A/PLIN6-A and PLIN1-C/PLIN3-T is indicative of decreased risk of obesity and obesity-related diseases in the individual of Malayan descent.

In one embodiment, a method of determining a decreased risk of obesity and obesity-related diseases in an individual of Indian descent is provided comprising the steps of: a) genotyping the PLIN1 6209T/C, PLIN3 10171A/T, PLIN4 11482G/A, PLIN5 13041A/G, PLIN6 14995 A/T loci from a biological sample taken from the individual; b) creating a haplotype based on the PLIN genotypes as determined in step (a); and c) correlating the haplotype with the ethnic background of the individual, wherein a haplotype selected from the group of consisting of PLIN1-C/PLIN3-A/PLIN4-G/PLIN5-A/PLIN6A; PLIN1-C/PLIN3-A/PLIN4-G/PLIN5-A/PLIN6-A; and PLIN1-C/PLIN3-C is indicative of decreased risk of obesity and obesity-related diseases in the individual of Indian descent.

In one embodiment, the individual whom a decreased risk of obesity and obesity-related diseases is assessed is a woman.

The invention further provides for a kit comprising primer pairs to amplify nucleic acid regions covering PLIN1 6209T/C, PLIN3 10171A/T, PLIN4 11482G/A, PLIN5 13041A/G, and PLIN6 14995 A/T polymorphisms and instructions including the haplotypes associated with increased or decreased risk of obesity and their correlation with an ethnic group.

In one embodiment, the kit comprises primer pairs of SEQ ID NO: 1 and SEQ ID NO: 2 to amplify nucleic acid region covering PLIN1 polymorphism; SEQ ID NO: 7 and SEQ ID NO: 8 to amplify nucleic acid region covering PLIN3 polymorphism; SEQ ID NO: 10 and SEQ ID NO: 11 to amplify nucleic acid region covering PLIN4 polymorphism; SEQ ID NO: 13 and SEQ ID NO: 14 to amplify nucleic acid region covering PLIN5 polymorphisms; and SEQ ID NO: 16 and SEQ ID NO: 17 to amplify nucleic acid region covering PLIN6 polymorphisms, and instructions including the haplotypes associated with increased or decreased risk of obesity and their correlation with an ethnic group.

### BRIEF DESCRIPTION OF FIGURES

FIG. 1 shows the nomenclature of the PLIN polymorphisms. Positions of the polymorphisms examined in the present study are indicated as vertical short lines, with the names under them. The square above the gene diagram shows the sequence encompassing nucleotide denoted “+1” in our nomenclature. The A of the ATG of the initiator Methionine codon is indicated as bold Italic letter, with its genomic position on the reference sequence (GenBank accession No. GI21431190) labeled above. The corresponding amino acids are also illustrated. The square with slash line indicates the region where alternative splicing may occur.

FIG. 2 shows the BMI for the combined genotypes of the PLIN1 and PLIN4 SNPs after controlling for PLIN5 and PLIN6 in women from sample 1. Age-adjusted means; error bars: SEM.

FIG. 3 shows the BMI for the combined genotypes of the PLIN5 and PLIN6 SNPs after controlling for PLIN1 and PLIN4 in women from sample 1. Age-adjusted means; error bars: SEM.

FIGS. 4A and 4B show a graphics of the results of weight gain or loss in women with PLIN4 wild type allele 1 and carriers of the PLIN4 allele 2 after dieting. Graphs clearly indicate that women with the heterozygous PLIN4 allele 2 are much more prone to gain weight if they do not continue on the diet.

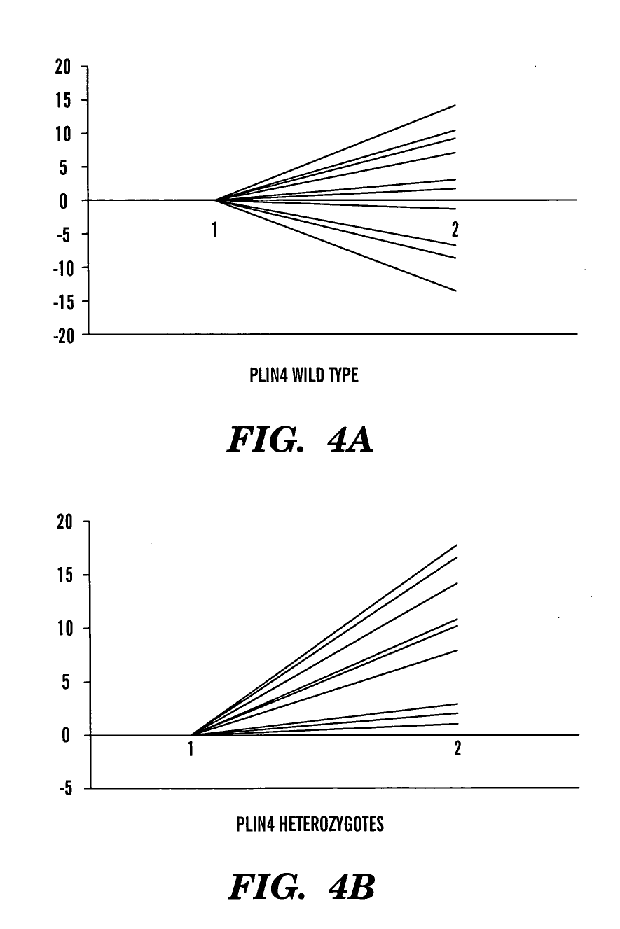

FIG. 5 shows a chart of the LD matrix in the study population. Pairwise LD measures (D′) between the four genotyped PLIN SNPs (6209C>T, 1 1482G>A, 13041A>G, and, 14995A>T) are displayed above the diagonal, while the corresponding P values are presented below the diagonal.

FIG. 6 shows a graph illustrating differences in body fatness measures (BMI, percent body fat, and waist) and standard errors between genotypes at the PLIN 13041A>G and 14995A>T SNPs in women. For the PLIN 13041A>G SNP. 11=AA, 12=AG and 22=GG. For the 14995A>T SNP, 11=AA, 12=AT and 22=TT.

FIG. 7 shows a chart of the LD matrix by ethnics in Singapore. Pairwise LD measures (D′) between the five genotyped PLIN SNPs (6209C>T, 10171A>T, 1 1482G>A, 13041A>G, and, 14995A>T) were displayed above the diagonal, while the corresponding P values were presented below the diagonal.

FIG. 8 shows a graph of the odds ratio (OR) for various PLIN5. Multivariate ORs and 95% CIs for obesity (BMI≧30 kg/m2) for PLIN 11482G>A, 13041A>G, and 14995A>T in Malays and Indians. For each SNP, the genotype group with wild type homozygotes and the heterozygotes was used as reference. OR was obtained by comparing homozygous variation with the reference.

### DETAILED DESCRIPTION OF THE INVENTION

The present invention is directed to new genetic variants or polymorphisms at the perilipin locus (PLIN) including PLIN1: 6209T (allele 1)>C (allele 2); PLIN3 10171 (allele 1) A>T (allele 2); PLIN4: 11482G (allele 1)>A (allele 2); PLIN5: 13041A (allele 1)>G (allele 2) and PLIN6: 14995A (allele 1)>T (allele 2), and their use in diagnostic and prognostic applications for obesity and related metabolic diseases as well as their use in treatment of obesity and related metabolic disorders. Sequence numbers referred to are in accordance with the GenBank sequence ID No. gi21431190.

The invention is directed to a novel PLIN haplotype which is associated with lower body mass index (BMI) and is therefore protective of obesity and related metabolic diseases, such as cardiovascular disease as well as PLIN haplotypes, which are associated with an elevated BMI and are therefore a risk factor of obesity and related metabolic diseases, such as cardiovascular disease and metabolic syndrome.

As used herein, “an individual of Mediterranean descent” refers to people who have a ancestors from the geographic region of the Mediterrania including but not limited to Spain, France, Italy, and Portugal. Preferably, at least one ancestor is from the geographic region of the Mediterrania.

As used herein, “an individual of Caucasian descent” refers to people who have ancestors from the geographic region of Northern, Eastern, or Central Europe. Generally the individuals have light skin color and are from regions including, but not limited to, North America, England, Russia, and Germany. Preferably, at least one ancestor is from Northern, Eastern, or Central Europe.

As used herein, “an individual of Malayan descent” refers to people who have ancestors from the geographic region of Malaysia and surrounding areas including, but not limited to, Malaysia, Indonesia, Brunei, and Singapore. Preferably, at least one ancestor is from Malaysia or surrounding areas.

As used herein, “an individual of Indian descent” refers to people who have a have ancestors from the geographic region India and surrounding areas including, but not limited to, India, Pakistan, Nepal and Bangladesh. Preferably, at least one ancestor is from India or surrounding areas.

Cardiovascular diseases (CVD) or diseases of the circulatory system represent various clinical conditions due to atherosclerotic impairment of coronary, cerebral or peripheral arteries. CVD are considered nowadays as the major cause of death in developed countries for men and women. Detailed epidemiological data for CVD are available from the American Heart Association's “2002 Heart and Statistical Update” summarizing the risk factors. 61,800,000 Americans suffer from one or more types of CVD (Rational diagnosis of cardiovascular disease, Müller M M, Griesmacher A, eJIFCC Vol 14 no 2: http://www.ifcc.org/ejifcc/vol14no2/1402062003012n.htm). There are presently several markers to diagnose an acute cardiovascular disease including use of a so-called “early” and a “late” marker released from cardiac myocytes under ischaemic conditions such as myotropin and cardiac troponins (Id.).

Metabolic syndrome is characterized by a group of metabolic risk factors in one person. These include a) central obesity (excessive fat tissue in and around the abdomen), b) atherogenic dyslipidemia (blood fat that foster plaque buildups in artery walls), c) raised blood pressure (130/85 mmHg or higher), d) insulin resistance or glucose intolerance, e) a prothrombotic state (e.g., high fibrinogen or plasminogen activator inhibitor −1 in the blood and f) a proinflammatory state (e.g., elevated high-sensitivity C-reactive protein in the blood). The underlying causes of this syndrome are overweight/obesity, physical inactivity and genetic factors. People with the metabolic syndrome are at increased risk of coronary heart disease, other diseases related to plaque buildups in artery walls (e.g., stroke and peripheral vascular disease) and type 2 diabetes.

In one embodiment, the present invention provides a novel means to assess susceptibility for cardiovascular diseases and metabolic syndrome by determining the PLIN haplotypes in an individual.

Perilipin (PLIN) is a hormonally-regulated phosphoprotein that encircles the lipid storage droplet in adipocytes (Greenberg, A. S.; Egan, J. J.; Wek, S. A.; Takeda, T.; Londos, C.; Kimmel, A. K. (Abstract) Clin. Res. 39: 287A only, 1991). It is the major cellular A-kinase substrate in adipocytes that coats intracellular lipid droplets and modulates adipocyte lipolysis activity. Nishiu et al. cloned a cDNA encoding human perilipin from an adipose tissue cDNA library (Genomics 48: 254-257, 1998; GenBank Nucleic Acid ID No. gi:3041770). The human gene encodes a 522-amino acid polypeptide that is 79% identical to the rat homolog isolated by Greenberg et al. (Proc. Nat. Acad. Sci. 90: 12035-12039, 1993).

The present invention is based upon identification and evaluation of the associations of several novel genetic variants at the perilipin locus (PLIN) with obesity and related metabolic disorders as well a cardiovascular disease, the variants including PLIN1: 6209T>C; PLIN3: 10171A>T; PLIN4: 11482G>A; PLIN5: 13041A>G and PLIN6: 14995A>T.

We determined associations of the PLIN polymorphisms and haplotypes in 788 males and 801 females randomly selected from Mediterranean population (sample 1), and 157 hospitalized obese subjects (sample 2). Surprisingly, in the whole population, the less common alleles of perilipin, namely PLIN1 allele 2 and PLIN4 allele 2 were significantly associated with reduced risk of obesity in women (OR=0.65, 95% CI: 0.48-0.88 and OR=0.60, 95% CI: 0.44-0.83, respectively). We also surprisingly found that in women from sample 1, the less common alleles of PLIN1 and PLIN4 were significantly associated with lower BMI as compared with the wild-type, i.e. the allele 1. In these women, PLIN4 was also associated with lower waist-to-hip ratio, fasting glucose, and plasma triacylglycerol concentrations. Haplotype analysis confirmed these results and revealed synergic effects of PLIN1 and PLIN4 on BMI in all women. No statistically significant associations were found in men from sample 1. Nonetheless, in obese men, carriers of the less common allele 2 of PLIN4 had significantly lower BMI than non-carriers. In both obese men and women the less common allele of PLIN1 and PLIN4 were associated with higher plasma glucose, and differed from sample 1 (P for interactions<0.05). Therefore, our data indicate that PLIN-2/PLIN4-2 haplotype is a protective obesity-susceptibility haplotype and has implication for the development of the metabolic syndrome and cardiovascular disease.

Therefore, in one embodiment, the invention provides a method of assessing an individual's predisposition to obesity and obesity-related diseases in an individual. The method comprises identifying and analyzing the PLIN polymorphisms in an isolated nucleic acid sample taken from the individual wherein presence of PLIN1 allele 1 and PLIN4 allele 1 together in the same chromatid in the nucleic acid sample (e.g. PLIN1-1/PLIN4-1 haplotype) indicates genetic predisposition to obesity and related metabolic diseases in the individual. Preferably the individual is of Mediterranean or Caucasian descent.

In one embodiment, the invention provides a method of assessing an individual's predisposition to cardiovascular disease wherein the method comprises identifying and analyzing the PLIN polymorphisms in an isolated nucleic acid sample taken from the individual, wherein presence of PLIN1 allele 1 and PLIN4 allele 1 in the same chromatid in the nucleic acid sample (e.g. PLIN1-1/PLIN4-1 haplotype) indicates predisposition to cardiovascular disease. Preferably the individual is of Mediterranean or Caucasian descent.

Alternatively, in one embodiment the invention provides a method of identifying individuals who are less likely to gain weight and who, after dieting, can be expected to better keep the reduced weight. The method comprises analyzing the isolated nucleic acids from an individual for the PLIN alleles, wherein the presence of allele 2 of the PLIN1 and PLIN4 indicate presence of obesity protective genotype in the individual. Preferably the individual is of Mediterranean or Caucasian descent.

The invention further provides haplotypes useful in diagnosing an individual at risk of developing obesity and/or obesity related diseases, including, but not limited to cardiovascular disease. One of these haplotypes consist of the polymorphisms including PLIN1; PLIN4; PLIN5; and PLIN6. Accordingly, haplotype 1111 consists of alleles 1 in all the above-identified loci, and haplotype 2222 consists of alleles 2 in all the above-identified loci. The haplotype 2211 in the nucleic acid sample from an individual, preferably a woman, indicates that the individual has decreased risk for developing obesity and/or cardiovascular disease. Conversely, an individual with haplotypes 1122 or 1111, has increased risk for developing obesity and/or cardiovascular disease. Preferably, when using these haplotypes for prognosis and or diagnosis, the individual is of Caucasian or Mediterranean descent.

In yet another embodiment, the invention provides a method of identifying an individual at risk of re-gaining weight after dieting. The method comprises analyzing the PLIN4 locus in the nucleic acid sample from the individual, wherein the presence of allele 2 in either one or both alleles of the PLIN4 locus is indicative of increased risk of regaining weight.

We also determined associations of the individual polymorphisms in the various PLIN loci and the PLIN haplotypes in a multi-ethnic Asian population. We examined five common single nucleotide polymorphisms (SNPs) at the Perilipin (PLIN) loci PLIN1, PLIN3, PLIN4, PLIN5 and PLIN6, wherein the polymorphisms were: PLIN 6209C>T, 10171 A>T, 11482G>A, 13041A>G, and 14995A>T respectively. We investigated their association with obesity risk and other variables related to the metabolic syndrome. The study population involved 4,131 subjects of three ethnic groups (Chinese, Malay, and Indian) from Singapore. Analysis indicated that haplotype 11212 was shared by both Malays and Indians and was significantly associated with increased obesity risk as compared to the most common haplotype 21111 (OR=1.65, 95% CI 1.11-2.46 for Malays, and OR=1.94, 95% CI 1.06-3.53 for Indians). Haplotype analyses using a subgroup of SNPs (11482G>A, 13041A>G, and 14995A>T) in positive LD with each other revealed that haplotypes 212 (OR=2.04, 95% CI 1.28-3.25) and 222 (OR=2.05, 95% CI 1.35-3.12) were associated with increased obesity risk in Malays, and, haplotype 212 (OR=2.16, 95% CI 1.10-4.26) was significantly associated with increased obesity risk in Indians, after adjusting for covariates including age, sex, smoking, alcohol consumption, exercise, and diabetes status. Individual SNP analyses demonstrated that covariate adjusted, the PLIN 14995A>T SNP was significantly associated with increased obesity risk in both Malays (OR=2.28, 95% CI 1.45-3.57) and Indians (OR=2.04, 95% CI 1.08-3.84). Whereas the PLIN 11482G>A ((OR=1.94, 95% CI 1.22-3.08) and the PLIN 13041A>G (OR=1.87, 95% CI 1.08-3.25) were associated with increased obesity risk only in Malays.

Therefore, in one embodiment, the invention provides a method of assessing an increased risk of developing obesity-related diseases in an individual of Malayan or Indian descent. The method comprises identifying and analyzing the PLIN polymorphisms in an isolated nucleic acid sample taken from the individual wherein halotype PLIN4-2/PLIN6-2, i.e., presence of PLIN4 allele 2 and PLIN6 allele 2 together in the same chromatid in the nucleic acid sample indicates risk of developing obesity and related diseases in the individual.

In one embodiment, the invention provides a method of assessing the predisposition to cardiovascular disease in an individual of Malayan or Indian descent, wherein the method comprises identifying and analyzing the PLIN polymorphisms and haplotypes in an isolated nucleic acid sample taken from the individual, wherein presence of a haplotype PLIN4-2/PLIN6-2 i.e., PLIN4 allele 2 and PLIN6 allele 2 together in the same chromatid in the nucleic acid sample indicates predisposition to cardiovascular disease.

In another embodiment, the invention provides a method of assessing a predisposition to obesity and obesity-related diseases in either an individual that is of Malayan or Indian descent wherein the method comprises identifying and genotyping the PLIN6 locus in an isolated nucleic acid sample taken from the individual wherein the presence of homozygosity for the T allele (allele 2) at PLIN6 indicates an increased risk of obesity and related diseases in the individual of Malayan or Indian descent.

In another embodiment, the invention provides a method of assessing a predisposition to obesity and obesity-related diseases in either an individual that is of Malayan or Indian descent wherein the method comprises identifying and genotyping the PLIN4 locus in an isolated nucleic acid sample taken from the individual wherein the presence of homozygosity for the A allele (rare allele) at PLIN4 indicates an increased risk of obesity and related diseases in the individual of Malayan or Indian descent.

In another embodiment, the invention provides a method of assessing a predisposition to obesity and obesity-related diseases in either an individual that is of Malayan or Indian descent wherein the method comprises identifying and genotyping the PLIN5 locus in an isolated nucleic acid sample taken from the individual wherein the presence of homozygosity for the G allele (rare allele) at PLIN5 indicates an increased risk of obesity and related diseases in the individual of Malayan or Indian descent.

The invention further provides for haplotypes useful in diagnosing Malays or Indians at increased risk of developing obesity and/or obesity related diseases. One haplotype consists of the polymorphisms including PLIN1; PLIN3; PLIN4; PLIN5; and PLIN6. Accordingly, haplotype 11111 consists of alleles 1 in all the above-identified loci, and haplotype 22222 consists of alleles 2 in all the above-identified loci. A haplotype 11212 or 11222 in the nucleic acid sample from an individual of Malayan descent indicates that the individual is at an increased risk for developing obesity and/or cardiovascular disease. A haplotype of 11212 in a nucleic acid sample from an individual of Indian descent indicates that the individual is at an increased risk for developing obesity and/or cardiovascular disease. A haplotype of 12111 or 21111 in the nucleotide sample from an individual of Malayan descent is associated with a decreased risk of obesity. In addition, a haplotype of 21111 in the nucleotide sample from an Indian is associated with a decreased risk of obesity.

Another haplotype useful in diagnosing individuals of Malayan and Indian descent consists of the polymorphisms including PLIN4; PLIN5; and PLIN6. Accordingly, haplotype 111 consists of alleles 1 in all the above-identified loci, and haplotype 222 consists of alleles 2 in all the above-identified loci, wherein a haplotype of 212, 222, or 121 from an individual of Malayan descent indicates that the individual is at an increased risk for developing obesity and/or cardiovascular disease. A haplotype of 212, or 122 present in the nucleic acid sample from an individual of Indian descent indicates that the individual is at an increased risk for developing obesity and/or cardiovascular disease.

In a further embodiment, the invention provides a method of assessing a predisposition to obesity and obesity-related diseases in individuals of Malayan or Indian descent, wherein the method comprises genotyping PLIN1 and PLIN3 loci in the isolated nucleic acids from an individual and creating a phenotype comprising these 2 loci, wherein a haplotype PLIN1-1/PLIN-3/1 i.e., PLIN1 allele 1 and PLIN3 allele 1 together in the same chromatid indicates an increased risk for developing obesity and/or cardiovascular disease.

In another embodiment, the invention further provides a method of identifying in individuals of Malayan or Indian descent who are less likely to gain weight and who, after dieting, can be expected to better keep the reduced weight. The method comprises genotyping PLIN1 and PLIN3 loci in the isolated nucleic acids from an individual and creating a haplotype for the PLIN alleles, wherein the presence of a haplotype PLIN1-1/PLIN3-2 i.e., PLIN1 allele 1 and PLIN3 allele 2 together in the same chromatid indicates presence of obesity protective genotype in the individual.

We also performed a study to determine associations of the PLIN polymorphisms and haplotypes in individuals of Caucasian descent from the United States. Four PLIN SNPs (PLIN 6209T>C, 11482G>A, 13041A>G, and 14995A>T) were genotyped in 734 white subjects (373 men and 361 women) attending a residential lifestyle intervention program. Multivariate analysis demonstrated that, in women, two of the SNPs (13041A>G, and 14995A>T) were significantly associated with percent body fat (P=0.016 for 13041A>G and P=0.010 for 14995A>T) and waist circumference (P=0.020 for 13041A>G and P=0.045 for 14995A>T). Moreover, haplotype analysis using these two SNPs indicated that haplotype PLIN5-A/PLIN6-T and PLIN5-G/PLIN6-T were both associated with significantly increased obesity risk (OR=1.76, 95% CI 1.07-2.90 for haplotype PLIN5-A/PLIN6-T, and, OR=1.73, 95% CI 1.06-2.82 for haplotype PLIN5-G/PLIN6-T) when compared with haplotype PLIN5-A/PLIN6-A. No significant associations between PLIN variations and obesity were found in men. Thus, PLIN is a significant genetic determinant for obesity risk in Caucasians and women are more sensitive to the genetic effects of perilipin than men.

Therefore, in one embodiment, the invention provides a method of assessing an individual's predisposition to obesity and obesity-related diseases in individuals of Caucasian descent. The method comprises genotyping and haplotyping the PLIN polymorphisms in an isolated nucleic acid sample taken from the individual of Caucasian descent, wherein presence of a haplotype PLIN5-2/PLIN6-2 or PLIN5-1/PLIN6-2 in the nucleic acid sample indicates increased risk of developing obesity and related diseases in the individual. Preferably the individual is a woman.

In one embodiment, the invention provides a method of assessing the predisposition of an individual of Caucasian descent to cardiovascular disease wherein the method comprises genotyping and haplotyping the PLIN polymorphisms in an isolated nucleic acid sample taken from the individual of Caucasian descent, wherein presence of a haplotype PLIN5-2/PLIN6-2 or PLIN5-1/PLIN6-2 in the nucleic acid sample indicates increased risk of developing cardiovascular disease. Preferably the individual is a woman.

Alternatively, in one embodiment the invention provides a method of identifying individuals of Caucasian descent who are less likely to gain weight and who, after dieting, can be expected to better keep the reduced weight. The method comprises isolating nucleic acids from an individual, genotyping PLIN loci, wherein the presence of allele 1 of the PLIN5 and PLIN6 indicate presence of obesity protective genotype in the individual and is indicative of an individual who will more likely keep off weight after dieting. Preferably the individual is a woman.

The invention further provides haplotypes useful in diagnosing individuals of Caucasian descent who are at risk of developing obesity and/or obesity related diseases, including, but not limited to cardiovascular disease. One of these haplotypes consist of the alleles in loci PLIN1, PLN4, PLIN5 and PLIN6. Accordingly, haplotype 1111 consists of alleles 1 in all the above-identified loci, and haplotype 22222 consists of alleles 2 in the above-identified loci, wherein the haplotype of 1122 in the nucleic acid sample from the individual of Caucasian descent indicates that the individual is more susceptible to obesity and/or cardiovascular disease, and wherein the Caucasian with haplotype 2111 is less susceptible to developing obesity and/or cardiovascular disease (See Table 15).

The invention also provides novel PLIN polymorphisms, and oligonucleotides useful for analysis of the novel PLIN polymorphisms by amplifying across a single nucleotide polymorphic site of the present invention. The invention further provides oligonucleotides useful for sequencing said amplified sequence.

In one embodiment the primers for amplifying PLIN, PLIN2, PLIN3, PLIN4, PLIN5 and PLIN6 are the nucleic acid sequences depicted in SEQ ID NO: 1 and 2, SEQ ID NO: 4 and 5, SEQ ID NO: 7 and 8, SEQ ID NO: 10 and 11; SEQ ID NO: 13 and 14, and SEQ ID NO: 16 and 17, respectively.

The invention further provides the following novel polymorphisms: PLIN1: 6209 T (allele 1)>6209 C (allele 2); PLIN3 10171 (allele 1) A>T (allele 2); PLIN4: 11482 G (allele 1)>11482 A (allele 2); PLIN5: 13041 A>13041 G (allele 2) and PLIN6: 14995 A (allele 1)>14995 T (allele 2). See Chart below.

| Locus | Allele 1 | Allele 2 |
|---|---|---|
| PLIN1 | T | C |
| PLIN3 | A | T |
| PLIN4 | G | A |
| PLIN5 | A | G |
| PLIN6 | A | T |

Therefore, in one embodiment, the invention provides polymorphisms which are a risk factor propensity for weight gain and/or cardiovascular disease in Mediterranean individual. In one embodiment, the polymorphism is allele 1 of PLIN1 (6209 T). In another embodiment, the polymorphism is allele 1 of PLIN4 (11482 G).

In another embodiment, the invention provides polymorphisms which are a risk factor propensity for weight gain and/or cardiovascular disease in individuals of Caucasian descent. When identified as homozygotes in the PLIN loci, they are associated with increased risk of weight gain. In one embodiment, the polymorphism is allele G of PLIN5 (13041 G). In another embodiment, the polymorphism is allele T of PLIN6 (14995 T).

In still another embodiment, the invention provides a polymorphism which when present as a homozygous allele is a risk factor propensity for weigh gain and/or cardiovascular disease in individuals of Malayan or Indian descent. The polymorphism is allele 2 of PLIN6 (14995 T) locus, i.e., T/T in PLIN6 is a risk factor.

In another embodiment, the invention provides polymorphisms which are a risk factor propensity for weight gain and/or cardiovascular disease in individuals of Malayan descent. In one embodiment, the polymorphism is allele 2 of PLIN5 (13041 G). In still another embodiment, the polymorphism is allele 2 of PLIN4 (11482 A).

The invention further provides a diagnostic method for identifying individuals who are less prone to obesity and obesity related diseases comprising the steps of obtaining a nucleic acid sample from an individual, analyzing the isolated nucleic acids, genotyping the allele variants in the sample and creating a haplotype from the genotypes. Table 15 illustrates haplotypes that if present in a individual of the indicated ethnic group, indicate the individual is less prone to obesity and obesity related diseases. Haplotypes in Table 15 are read vertically, for example, haplotye (a) is PLIN5-A/PLIN6-A and haplotype (h) is PLIN1-C/PLIN3-A/PLIN4-G/PLIN5A/PLIN6A.

The invention further provides a diagnostic method for identifying individuals who are at an increased risk of obesity and obesity related diseases, such as cardiovascular disease. The method comprises the steps of obtaining a nucleic acid sample from an individual, analyzing the isolated nucleic acids, genotyping the allele variants in the sample and creating a haplotype from the genotypes. Table 16 illustrates haplotypes that, if present in a individual of the indicated ethnic group, indicate the individual is at an increased risk of developing obesity and obesity related diseases. Haplotypes in Table 16 are read vertically, for example, haplotye (k) is PLIN5-G/PLIN6-T and haplotype (w) is PLIN1-T/PLIN3-A/PLIN4-A/PLIN5A/PLIN6T.

In another embodiment, the invention provides a diagnostic method for identifying females at risk of developing obesity and obesity related diseases, such as cardiovascular disease, comprising the steps of obtaining a nucleic acid sample from a female individual, amplifying a sequence using appropriate PLIN-PCR primers for amplifying across a polymorphic site, detecting the allele variants in the sample, and analyzing the result.

Biological sample used as a source material for isolating the nucleic acids in the instant invention include solid materials (e.g., tissue, cell pellets, biopsies) and biological fluids (e.g. blood, saliva, amniotic fluid, mouth wash, urine). Nucleic acid molecules of the instant invention include DNA and RNA and can be isolated from a particular biological sample using any of a number of procedures, which are well-known in the art, the particular isolation procedure chosen being appropriate for the particular biological sample. Methods of isolating and analyzing nucleic acid variants as described above are well known to one skilled in the art and can be found, for example in the Molecular Cloning: A Laboratory Manual, 3rd Ed., Sambrook and Russel, Cold Spring Harbor Laboratory Press, 2001.

The PLIN polymorphisms of the present invention can be detected from the isolated nucleic acids using techniques including direct analysis of isolated nucleic acids such as Southern Blot Hybridization (DNA) or direct nucleic acid sequencing (Molecular Cloning: A Laboratory Manual, 3rd Ed., Sambrook and Russel, Cold Spring Harbor Laboratory Press, 2001).

An alternative method useful according to the present invention for direct analysis of the PLIN polymorphisms is the INVADER® assay (Third Wave Technologies, Inc (Madison, Wis.). This assay is generally based upon a structure-specific nuclease activity of a variety of enzymes, which are used to cleave a target-dependent cleavage structure, thereby indicating the presence of specific nucleic acid sequences or specific variations thereof in a sample (see, e.g. U.S. Pat. No. 6,458,535).

Preferably, a PCR based techniques are used. After PCR, the polymorphic nucleic acids can be identified using, for example direct sequencing with labeled primers, such as radioactively or fluorescently labeled primers; single-stand conformation polymorphism analysis (SSCP), denaturating gradient gel electrophoresis (DGGE); and chemical cleavage analysis, all of which are explained in detail, for example, in the Molecular Cloning: A Laboratory Manual, 3rd Ed., Sambrook and Russel, Cold Spring Harbor Laboratory Press, 2001.

The polymorphisms are preferably analyzed using methods amenable for automation such as the different methods for primer extension analysis. Primer extension analysis can be preformed using any method known to one skilled in the art including PYROSEQUENCING™ (Uppsala, Sweden); Mass Spectrometry including MALDI-TOF, or Matrix Assisted Laser Desorption Ionization—Time of Flight; genomic nucleic acid arrays (Shalon et al., Genome Research 6(7):639-45, 1996; Bernard et al., Nucleic Acids Research 24(8):1435-42, 1996); solid-phase mini-sequencing technique (U.S. Pat. No. 6,013,431, Suomalainen et al. Mol. Biotechnol. Jun.; 15(2):123-31, 2000); ion-pair high-performance liquid chromatography (Doris et al. J. Chromatogr. A May 8; 806(1):47-60, 1998); and 5′ nuclease assay or real-time RT-PCR (Holland et al. Proc Natl Acad Sci USA 88: 7276-7280, 1991), or primer extension methods described in the U.S. Pat. No. 6,355,433. Nucleic acids sequencing, for example using any automated sequencing system and either labeled primers or labeled terminator dideoxynucleotides can also be used to detect the polymorphisms. Systems for automated sequence analysis include, for example, Hitachi FMBIO® and Hitachi FMBIO® II Fluorescent Scanners (Hitachi Genetic Systems, Alameda, Calif.); Spectrumedix® SCE 9610 Fully Automated 96-Capillary Electrophoresis Genetic Analysis System (SpectruMedix LLC, State College, Pa.); ABI PRISM® 377 DNA Sequencer; ABI® 373 DNA Sequencer; ABI PRISM® 310 Genetic Analyzer; ABI PRISM® 3100 Genetic Analyzer; ABI PRISM® 3700 DNA Analyzer (Applied Biosystems, Headquarters, Foster City, Calif.); Molecular Dynamics FluorImager™ 575 and SI Fluorescent Scanners and Molecular Dynamics FluorImager™ 595 Fluorescent Scanners (Amersham Biosciences UK Limited, Little Chalfont, Buckinghamshire, England); GenomyxSC™ DNA Sequencing System (Genomyx Corporation (Foster City, Calif.); Pharmacia ALF™ DNA Sequencer and Pharmacia ALFexpress™ (Amersham Biosciences UK Limited, Little Chalfont, Buckinghamshire, England).

PCR, nucleic acid sequencing and primer extension reactions for one nucleic acid sample can be performed in the same or separate reactions using the primers designed to amplify and detect the polymorphic PLIN nucleotides.

In one embodiment, the invention provides a nucleic acid chip including the polymorphic PLIN1, PLIN3, PLIN4, PLIN5, and PLIN6 alleles for the screening of the individual with a risk of PLIN-associated obesity and/or obesity-related diseases, including cardiovascular disease, or PLIN-associated protection from obesity and/or obesity-related diseases, such as cardiovascular disease. Such a chip can include any number of other obesity-associated mutations and polymorphisms including but not limited to leptin, leptin receptor, MC4R and others. A list of obesity associated genes and polymorphisms can be found, for example, in Chagnon, Y. C., Pérusse, L., Weisnagel, S. J., Rankinen, T. and Bouchard, C. The Human Obesity Gene Map: The 1999 Update. Obesity Research 8 (1): 89-117, 2000, and on the web at http://www.obesity.chair.ulaval.ca/genemap.html.

Methods and techniques applicable to array synthesis have been described in U.S. Ser. No. 09/536,841, WO 00/58516, U.S. Pat. Nos. 412,087, 6,147,205, 6,262,216, 6,310,189, 5,889,165, and 5,959,098, 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285, which are all incorporated herein by reference in their entirety for all purposes. Additional methods of sample preparation and techniques for reducing the complexity of a nucleic sample are described, for example, in Dong et al., Genome Research 11, 1418 (2001), in U.S. Pat. Nos. 6,361,947, 6,391,592 and U.S. patent application Ser. Nos. 09/916,135, 09/920,491, 09/910,292, and 10/013,598.

Methods for conducting polynucleotide hybridization assays on the chips have been well developed in the art. Hybridization assay procedures and conditions will vary depending on the application and are selected in accordance with the general binding methods known including those referred to in: Maniatis et al. Molecular Cloning. A Laboratory Manual (2^{nd} Ed. Cold Spring Harbor, N.Y, 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described, for example, in U.S. Pat. Nos. 5,871,928, 5,874,219, 6,045,996 and 6,386,749, 6,391,623 each of which are incorporated herein by reference

Examples of methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Patent application 60/364,731 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001).

The present invention also makes use of various computer program products and software for a variety of purposes, such as probe design, management of data, analysis, and instrument operation. See, for example, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet.

The invention further provides for diagnostic kits. In one embodiment, the invention provides a kit comprising one or more primer pairs capable of amplifying the PLIN nucleic acid regions comprising the obesity associated polymorphic nucleotides of the present invention; buffer and nucleotide mix for the PCR reaction; appropriate enzymes for PCR reaction in same or separate containers as well as an instruction manual defining the PCR conditions, for example, as described in the Example below, as well as listing the obesity associated alleles and haplotypes as described in this specification. The kit may further comprise nucleic acid probes, preferably those listed on Table 1, either in dry form in a tube or a vial or in a buffer. In the preferred embodiment, these primers are the ones listed on Table 1. Primers may also be provided in the kit in either dry form in a tube or a vial, or alternatively dissolved into an appropriate aqueous buffer. The kit may also comprise primers for the primer extension method for detection of the specific PLIN polymorphisms as described above.

The kit also preferably includes a table listing the obesity risk haplotyes in various ethnic populations, such as Tables 15 and 16 as shown herein.

In one embodiment, the components of the kit are part of a kit providing for multiple obesity associated genes, polymorphisms and mutations known in to one skilled in the art.

A DNA haplotype, the phase determined association of several polymorphic markers (e.g., SNPs), is a statistically much more powerful method than the use of single markers alone for determining disease associations. Approaches for determining and identifying the haplotypes according to the present invention include a physical separation of homologous chromosomes via for example means of mouse cell line hybrid, cloning into a plasmid and allele specific PCR as well as computational determination of haplotypes.

According to the present invention, approaches that can be used to haplotype SNPs in the PLIN locus include, but are not limited to, single-strand conformational polymorphism (SSCP) analysis (Orita et al. (1989) Proc. Natl. Acad. Sci. USA 86:2766-2770), heteroduplex analysis (Prior et al. (1995) Hum. Mutat. 5:263-268), oligonucleotide ligation (Nickerson et al. (1990) Proc. Natl. Acad. Sci. USA 87:8923-8927) and hybridization assays (Conner et al. (1983) Proc. Natl. Acad. Sci. USA 80:278-282). Traditional Taq polymerase PCR-based strategies, such as PCR-RFLP, allele-specific amplification (ASA) (Ruano and Kidd (1989) Nucleic Acids Res. 17:8392), single-molecule dilution (SMD) (Ruano et al. (1990) Proc. Natl. Acad. Sci. USA 87:6296-6300), and coupled amplification and sequencing (CAS) (Ruano and Kidd (1991) Nucleic Acids Res. 19:6877-6882), are easily performed and highly sensitive methods to determine haplotypes of the present invention (Michalatos-Beloin et al. (1996) Nucleic Acids Res. 24:4841-4843; Barnes (1994) Proc. Natl. Acad. Sci. USA 91:5695-5699; Ruano and Kidd (1991) Nucleic Acids Res. 19:6877-6882).

In one embodiment, a long-range PCR (LR-PCR) is used to haplotype SNPs of the present invention. LR-PCR products are genotyped for SNPs using any genotyping methods known to one skilled in the art, and haplotypes inferred using mathematical approaches (e.g., Clark's algorithm (Clark (1990) Mol. Biol. Evol. 7:111-122).

In one embodiment, a haplotyping method useful according to the present invention is a physical separation of alleles by cloning, followed by sequencing. Other methods of haplotyping, useful according to the present invention include, but are not limited to monoallelic mutation analysis (MAMA) (Papadopoulos et al. (1995) Nature Genet. 11:99-102) and carbon nanotube probes (Woolley et al. (2000) Nature Biotech. 18:760-763). U.S. Patent Application No. US 2002/0081598 also discloses a useful haplotying method which involves the use of PCR amplification.

Computational algorithms such as expectation-maximization (EM), subtraction and PHASE are useful methods for statistical estimation of haplotypes (see, e.g., Clark, A. G. Inference of haplotypes from PCR-amplified samples of diploid populations. Mol Biol Evol 7, 111-22. (1990); Stephens, M., Smith, N. J. & Donnelly, P. A new statistical method for haplotype reconstruction from population data. Am J Hum Genet 68, 978-89. (2001); Templeton, A. R., Sing, C. F., Kessling, A. & Humphries, S. A cladistic analysis of phenotype associations with haplotypes inferred from restriction endonuclease mapping. II. The analysis of natural populations. Genetics 120, 1145-54. (1988)).

All the above-discussed methods are useful methods that can be employed in determining the haplotypes according to the methods of the present invention.

### EXAMPLES

### Example 1

### Gender-Specific Effects of PLIN Polymorphisms on Obesity-Related Variables in Individuals from the Eastern Mediterranean Coast of Spain

Materials and Methods

Subjects and Study Design

In total, 1746 white unrelated subjects were included in this report. The study population comprised 1589 individuals randomly selected from the Valencia Region on the Eastern Mediterranean coast of Spain (sample 1), and 157 obese subjects (sample 2), from the University General Hospital, located in the same geographical area. Briefly, sample 1 consisted of 788 men and 801 women, aged 18-85 years, who were chosen among individuals participating in a study aimed to ascertain the prevalence of both genetic and environmental cardiovascular risk factors in the Mediterranean Spanish population (14, 15). This sample comprised randomly selected workers, using a continuously updated computerized population register, as well as subjects randomly selected from the general population (15, 16). All these subjects were examined between 1999 and 2002. Sample 2, consisted of 29 men and 128 women aged 18-78 years, randomly selected from the Endocrinology Unit of the University General Hospital, Valencia, among those individuals referred consecutively for weight reduction treatment between 2001 and 2002. Baseline data were used for the present study. The study protocol was approved by the ethics committees of the Valencia University and the University General Hospital. All included subjects provided informed consent for participation and had both PLIN genotype available and data for the other variables examined. The mean age was 41.5±13.4 years for subjects from sample 1, and 47.0±13.7 years in sample 2. Cross-sectional, as well as case-control approaches, were applied in the statistical analyses. In the case-control approach, 438 subjects (157 from the Hospital and 281 from the general population) were classified as obese if their body mass index (BMI) was ≧30 Kg/m². The rest, 1308 subjects from the general population, were classified as non-obese.

Anthropometrical and Blood Pressure Measurements

Anthropometrical measurements were taken using standard techniques: weight with light clothing by digital scales; height without shoes by fixed stadiometer. BMI was calculated as weight (kg)/height (m²). Waist circumference was measured midway between the lower rib margin and the iliac crest in the horizontal plane. Hip circumference was measured at the point yielding the maximum circumference over the buttocks. Blood pressure was taken with a calibrated mercury sphygmomanometer following the WHO MONICA protocol with the average of two consecutive readings of the first and fifth Korotkoff sounds for systolic and diastolic blood pressure (SBP and DBP), respectively.

Biochemical, Clinical and Life-Style Data

Participants were instructed to fast for at least 12 hours before a morning examination. Venous blood was collected into EDTA-containing glass tubes. Plasma total cholesterol and TAGs were determined by a Technicon Chem 1 assay (Technicon Instruments, Tarrytown, N.Y.), and high-density lipoprotein cholesterol (HDL-C) was measured in the supernatant after precipitation of apolipoprotein B-containing lipoproteins with heparin-manganese chloride. Low-density lipoprotein cholesterol (LDL-C) was calculated according to the equation of Friedewald et al. (17) for samples with serum TAGs concentrations below 400 mg/dL. Fasting glucose was measured in fresh specimens with a hexokinase reagent kit.

Data on gender, date of birth, ethnicity, marital status, education, medication, health problems, history of type 2 diabetes, tobacco use, alcohol consumption and physical activity, were assessed by a self-administered questionnaire as previously reported.(14) Current smokers were defined as those smoking at least one cigarette per day. Alcohol consumption was carefully evaluated by a set of 22 questions about the use of alcoholic beverages during workdays and weekends. Physical activity was estimated from questions about regularly leisure-time physical sports, as well as the average number of hours per week spent in each activity. According to the type and time, subjects were categorized as sedentary (no physical exercise), moderate (one sport less than 3 hours/week) and high (one sport more than 3 hours/week or more than two sports per week). This variable was then dichotomized as sedentary (no physical exercise) versus active (moderate plus high). Education was classified into three categories: primary, secondary and university [including cycle I (3 years) and cycle II (5 years or more)] (14,15).

DNA Extraction and Genotyping

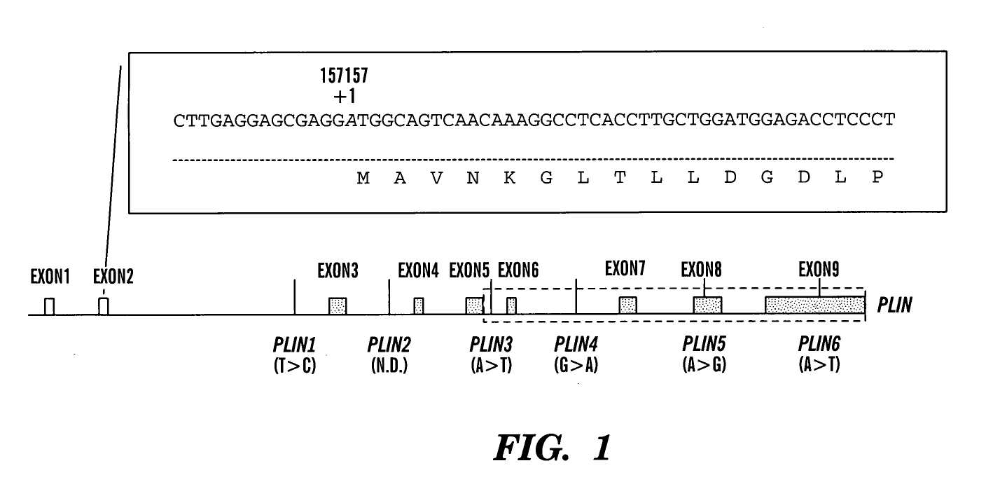

Genomic DNA was isolated from white blood cells by phenol-chloroform extraction and ethanol precipitation. The description and nomenclature for the six single nucleotide polymorphisms (SNPs) examined in this study are presented in FIG. 1 and Table 1. The polymorphisms were named according to the most recent recommendations (18). The reference sequence is GI21431190 (GenBank). Genotyping was carried out using Single Nucleotide Extension. First, the DNA fragments encompassing the 4 polymorphisms were amplified by multiplex polymerase chain reaction (PCR). The primers used are presented in Table 1. The PCR productions were 422 bp, 391 bp, 318 bp, 350 bp, 190 bp, and 469 bp for PLIN, PLIN2, PLIN3, PLIN4, PLIN5 and PLIN6, respectively. PCR amplification was carried out in a 1101 reaction volume containing 0.2 mmol/l of each dNTP, 0.2 μmol/l of each primer, 3.0 mmol/1 magnesium chloride, and 0.8 U of Qiagen Hotstar Taq polymerase. PCR cycling conditions were 95° C. for 10 min followed by 7 cycles of 95° C. for 30 seconds, 70° C. for 30 seconds, and 72° C. for 1 min, then followed by 41 cycles of 95° C. for 30 seconds, 65° C. for 30 seconds, and 72° C. for 1 min. A final extension phase of 2 min at 72° C. was included at the end of the protocol. The PCR products were incubated for 60 min at 37° C. with 2.5 U each of Exonuclease I (New England Biolabs, Inc. Beverly, Mass.) and Calf Intestinal Phosphatase (New England Biolabs, Inc. Beverly, Mass.) to remove un-incorporated dNTPs and primers. This was followed by incubation for 15 min at 75° C. to inactivate the enzymes.

Subsequently, Single Nucleotide Extension was carried out using the ABI Prism SnaPshot multiplex system (Applied Biosystems, Foster City, Calif.). Probes used for Single Nucleotide Extension are listed in Table 1. The extension reaction was carried out using PCR thermocycler in a 5 μl reaction mixture containing 1.5 μl of the Snapshot Ready Reaction Mastermix (Applied Biosystems, Foster City, Calif.), 1.0 μl of water, and 1.5 μl of multiplex PCR products and 1.0 μl of the probe mixture (1.5 μmol/l for PLIN1, PLIN2, PLIN3, and PLIN4, and 2.0 μmol/l for PLIN5 and PLIN6). The reaction conditions were 35 cycles of 96° C. for 30 seconds, 50° C. for 30 seconds, and 60° C. for 30 seconds. The reaction products were incubated for 60 min at 37° C. with 3 U Calf Intestinal Phosphatase to remove un-incorporated dNTPs, followed by incubation for 15 min at 75° C. to inactivate the enzyme. Genotyping was carried with the final products on an ABI Prism 3100 genetic analyzer (Applied Biosystems, Foster City, Calif.) using Genotyper version 3.7 (Applied Biosystems, Foster City, Calif.).

Statistical Analysis

Allele frequencies were estimated by gene counting, and 95% confidence intervals (CI) were calculated. x² tests (Pearson, Fisher exact test, or the Monte Carlo approach) were used to test differences between observed and expected frequencies, assuming Hardy-Weinberg equilibrium, to test linkage disequilibrium, and to test differences in percentages. Pairwise linkage disequilibrium coefficients were estimated by the LINKAGE program. D and D′ (D/Dmax) coefficients were calculated. Haplotypes were estimated by the EH program which uses the expectation-maximation algorithm to obtain maximum-likelihood estimates of the haplotype frequencies. Normal distribution for all continuous variables was checked. Triglycerides were logarithmically transformed to improve normality. Parametric test were applied to compare means. In addition, when the number of cases in each subgroup was very small, nonparametric tests (Mann-Whitney or Kruskal-Wallis) were applied. Multivariate linear regression analysis with dummy variables for categorical terms was used to test the null hypotheses of no association between genetic variants and obesity-related phenotypes. These statistical models allowed us to estimate the association of the genetic polymorphism with each dependent variable (obesity-related phenotypes) after adjustment for covariates. The main covariates were sex, age, BMI or life-style factors (tobacco smoking, alcohol consumption, physical activity, and education). Regression coefficients and adjusted means for each predictor were estimated from the models. Homogeneity of allelic effects according to gender or to the genetic or environmental factors was tested by introducing the corresponding terms of interaction in the more parsimonious linear regression model. Standard regression diagnostic procedures were used to ensure the appropriateness of these models. In the categorical analysis, obesity was defined dichotomously as BMI≧30 kg/m². Logistic regression models were fitted to estimate the risk:odds ratio (OR) and 95% confidence interval (CI) of obesity associated with the presence of each genetic variant as compared with the wild-type. Multiple logistic regression models with and without interaction terms were also fitted to control for the effect of covariates and effect modifiers. Association analyses were done using the SPSS, version 10.0 for windows.

Results

Identification of Novel Polymorphism, Frequencies and Linkage Disequilibrium

We used two different strategies to search for polymorphisms at the PLIN locus (FIG. 1). First, we sequenced the 5′ region of the PLIN gene in 40 unrelated subjects to search for common mutations potentially involved on the regulation of the PLIN gene. We concentrated on those regions that were significantly conserved between human and murine sequences (21). These analyses did not reveal any common mutation within the regions examined. Our second approach was based on searching for common polymorphisms in one of the public SNP database (http://www.ncbi.nlm.nih.gov/SNP/snp_ref.cgi?locusId=5346). We selected initial targets based on the following criteria: 1) SNP in exons were preferred over those in introns; 2) if several SNPs cluster in a narrow region, only one of them was selected. Six reported SNPs were initially selected (Table 1), two of them (PLIN2 and PLIN3) were not polymorphic and our analyses were based on the other four SNPs (PLIN, PLIN4, PLIN5 and PLIN6).

Table 2 shows demographic, biochemical and life-style characteristics of the 1746 unrelated subjects examined in this study: 1589 from the general population (sample 1), and 157 hospitalized morbidly obese patients (sample 2). In sample 1, the range of BMI was 16.2 to 52.5 Kg/m², with only 4% of subjects having a BMI≧35 Kg/m². In sample 2, the range of BMI was 30.1 to 79.1 Kg/m², with 88% of subjects having a BMI≧35 Kg/m². PLIN genotypes, allele frequencies and linkage disequilibrium coefficients for population sample 1 are given in Table 3. Genotype distributions did not deviate from Hardy-Weinberg expectations. As differences by gender in the genotype distributions were not significant for any polymorphism, data for men and women were pooled, and allele frequencies and pairwise linkage disequilibrium parameters were estimated for the whole sample. Allele 2 (G) at the PLIN5 locus was the most prevalent gene variant in sample 1 (allele frequency: 0.385; 95% CI 0.368 to 0.402); whereas allele 2 (A) at the PLIN4 locus was the less prevalent (allele frequency: 0.262; 95% CI 0.247 to 0.278). The strongest pairwise linkage disequilibrium was found between the PLIN1 polymorphism and the PLIN4 polymorphisms (D′: 0.958; p<0.001). Despite being statistically significant, much lower positive linkage disequilibrium was observed between the other polymorphisms, with D′ coefficients ranging from 0.453 to 0.149 (Table 3). Prevalence and linkage disequilibrium between the PLIN polymorphism in sample 2 were not different from sample 1. Likewise, genotype distributions in sample 2 were not different between men and women. The frequencies for the less common allele of the PLIN1, PLIN4, PLIN5, and PLIN6 polymorphism in sample 2 were: 0.37 (0.32-0.43); 0.24 (0.19-0.29); 0.40 (0.35-0.46) and 0.38 (0.33-0.46), respectively. However, the small sample size of this group largely affects the random error of these estimations. Thus, haplotypes were only estimated from all genotyped individuals in sample 1 (Table 4). All of the 16 possible four-polymorphism haplotypes were estimated to be present in this Mediterranean population. The haplotype consisting of the most frequent alleles at each polymorphism (“6209T/1482G/13041A/14995A”; further referred to as “1111”) was the most prevalent, with a relative frequency of 0.388. Of the 15 remaining haplotypes, only 4 had an allele frequency higher than 0.08, including the haplotype consisting of the least frequent alleles of each polymorphism (“6209C/11482A/13041G/14995T”; further referred to as “2222”).

Association Between the PLIN Polymorphisms and Obesity-Related Phenotypes. Single Polymorphism Genotype Analysis.

We next examined the association between the PLIN polymorphism and obesity-related variables. Considering the clinical and life-style differences between sample 1 and sample 2, the association analyses were performed separately for subjects from the general population and for obese patients. In order to increase the statistical power and after having verified the presence of an allelic effect compatible with a dominant, or at least, a co-dominant model of inheritance, individuals were classified as homozygotes for the most common allele or as carriers of the less common allele (1/2+2/2) for each polymorphism.

Associations in Sample 1

First, we evaluated the homogeneity of the genetic effect by gender and demonstrated several significant interactions. Therefore, we analyzed each gender separately. Table 5 shows age-adjusted means for BMI and other obesity-related variables in men from sample 1 according to the carrier status of the allele 2 variant within each of the four PLIN polymorphisms. We did not find significant differences between genotype groups regarding BMI, weight, waist-to-hip ratio, glucose, total cholesterol, HDL-C, LDL-C, TAGs and blood pressure. However, we found that in women from sample 1 BMI differed significantly between genotypes for both the PLIN1 and the PLIN4 polymorphisms, with the allele 2 being associated with lower BMI (Table 6). Mean values for BMI were 26.3±0.3 Kg/m² in 1/1 homozygotes vs 25.3±0.2 Kg/m² in women carrying the allele 2 for the PLIN1 polymorphism (p=0.004); and 26.1±0.2 Kg/m² in 1/1 homozygotes vs 25.2±0.3 Kg/m² in carriers of the allele 2 for the PLIN4 polymorphism (p=0.004). Likewise, carriers of the allele 2 at the PLIN1 locus weighted significantly less (p=0.007) than women homozygotes for the wild type genotype. The same was true for carriers of the less frequent allele at the PLIN4 locus (p=0.01). In addition, women carriers of the allele 2 for the PLIN4 polymorphism showed lower waist-to-hip ratio (p=0.032), lower fasting glucose (p=0.008) and lower plasma TAG concentrations (p=0.005) as compared with 1/1 homozygotes. Similar differences were found for the PLIN1 polymorphism, with borderline P values of 0.090 for fasting glucose, and 0.099 for TAGs. Both SNPS (PLIN1 and PLIN4) demonstrated significant gene-gender interactions determining BMI and body weight. In addition, for the PLIN4 polymorphism we found significant gene*gender interactions in determining waist-to-hip ratio (p=0.023) and TAGs (p=0.009). No significant gene*gender interactions were detected neither for the PLIN5 polymorphism nor for the PLIN6 polymorphism.

Carriers and non-carriers of the allele 2 for each polymorphism were not significantly different with respect to tobacco smoking, alcohol consumption, education, physical activity and diabetes in both men and women (results not shown). Therefore, differences found for the PLIN1 and the PLIN4 polymorphisms remained statistically significant even after adjustment for these potential confounders (p=0.012 and p=0.020 for BMI and weight for the PLIN1 polymorphism; p=0.014, p=0.029, p=0.046, p=0.003 and p=0.042 for BMI, weight, waist-to-hip ratio, glucose and TAGs, respectively for the PLIN4 polymorphism). Additional adjustment for BMI and medication did not modify the significance of the associations between fasting glucose and plasma lipids and PLIN4 genotypes [116.4±1.3 mg/dL in non carriers vs. 113.7±1.7 mg/dL in carriers of the allele 2 (p=0.010)]. However, differences in TAG concentrations were not statistically significant (p=0.327).

Associations in Sample 2

When we performed similar association analyses in the group of morbidly obese subjects (sample 2), a decrease in BMI associated with the allele 2 in the PLIN1 and the PLIN4 polymorphisms was detected in both men and women. This decrease was higher and statistically significant in men carrying the allele 2 in the PLIN4 polymorphism. In contrast with results observed in men from the general population, in this group of mainly morbidly obese men, the PLIN SNPs were associated with dramatic differences in BMI. Thus, for PLIN4, the age-adjusted means of BMI were 45.9±1.9 Kg/m2 in non-carriers vs. 35.6±1.3 Kg/m² in men carriers of the 2 allele (p=0.001). Likewise, adjusted-means for weight were 141.3±6.0 Kg in non-carriers vs. 107.9±6.3 Kg, in carriers of the 2 allele (p=0.001). Despite the small number of cases, these results in obese men were consistent and statistically significant in parametric, as well as in nonparametric tests. In obese women from sample 2, the decrease in BMI and weight observed in carriers of the allele 2 for the PLIN4 polymorphism was similar to that observed in women from the general population, however, because the lower number of women in this group, the difference did not reach the statistical significance [the age-adjusted means were: 43.1±0.9 Kg/m² vs. 41.1±6.3 Kg/m² (p=0.199) and 108.2±2.1 Kg vs. 102.4±2.9 Kg (p=0.112) in non carriers vs. carriers of the allele 2 of the PLIN4 SNP]. Further multivariate adjustment for tobacco smoking, alcohol consumption, education, physical activity, and diabetes did not affect the statistical significance of these results. Despite the decrease in BMI associated with the allele 2 in obese subjects, TAG concentrations did not differ significantly by genotype. Moreover, in these subjects, carriers of the allele 2 for the PLIN4 polymorphism showed higher plasma glucose concentrations than non-carriers. This effect was noted in both men and women, and differed from that observed for the same allele in subjects from the general population. Thus, in men from sample 2 plasma fasting glucose concentrations were 94.5±7.9 mg/dL vs. 117.1±7.7 mg/dL in non-carriers vs. carriers of the PLIN4 2 allele (P for interaction: PLIN4*obese=0.028), whereas in men from sample 1, no differences were noted. Conversely, in women from the general population, a decrease of plasma glucose associated with the allele 2 was found, whereas in women from sample 2, an increase in plasma glucose concentrations was observed (102.4±3.5 mg/dL vs. 108.2±3.9 mg/dL in non carriers vs. carriers of the PLIN4 2 allele). Statistically significant interaction terms were also obtained for PLIN1, PLIN5 and PLIN6 polymorphism with obesity in determining fasting glucose concentrations.

Association of PLIN Haplotypes with Metabolic Syndrome-Related Variables

We also evaluated the effect of PLIN haplotypes on several variables associated with the risk of metabolic syndrome (BMI, TAGs and fasting glucose). Eleven of the 16 possible haplotypes occurred with a very low relative frequency (below 5%). Therefore, we used a pseudohaplotype approach by comparing the effect of the homozygosity for the most common haplotype with the effect of a selected combination of genotypes, depending on their frequency and the specific association analysis carried out. First, results from Tables 5 and 6 were adjusted for the corresponding confounding effect of the other polymorphism by including these variables as control factors in the multiple regression models. Considering the higher association between PLIN1 and PLIN4, these variables were not simultaneously adjusted by each other in order to avoid the multicollinearity bias. Thus, PLIN1 and PLIN4 associations were adjusted for PLIN5 and PLIN6 polymorphisms, PLIN5, for PLIN4 and PLIN6, and PLIN6 for PLIN4 and PLIN5. The association between the PLIN1 polymorphism and BMI in women remained statistically significant after these adjustments (p=0.002). Moreover, the borderline statistical significant association of the PLIN1 polymorphism with fasting glucose in women, reached the statistical significance after adjustment for the PLIN6 polymorphism (p=0.032), and a slight decrease in the values for triglycerides were found after adjustment for PLIN5 (p=0.056) and PLIN6 (p=0.085). Likewise, the independent effect of the PLIN4 polymorphism in women were confirmed after adjustment for PLIN5 and PLIN6 polymorphisms and the associations previously reported in Table 6, remained statistically significant after these adjustments (p=0.023; p=0.015; p=0.035 for BMI, fasting glucose and TAGs, respectively after simultaneous adjustment for PLIN5 and PLIN6. In men, no significant variations were detected when results of Table 5, were adjusted for the additional genetic variants.

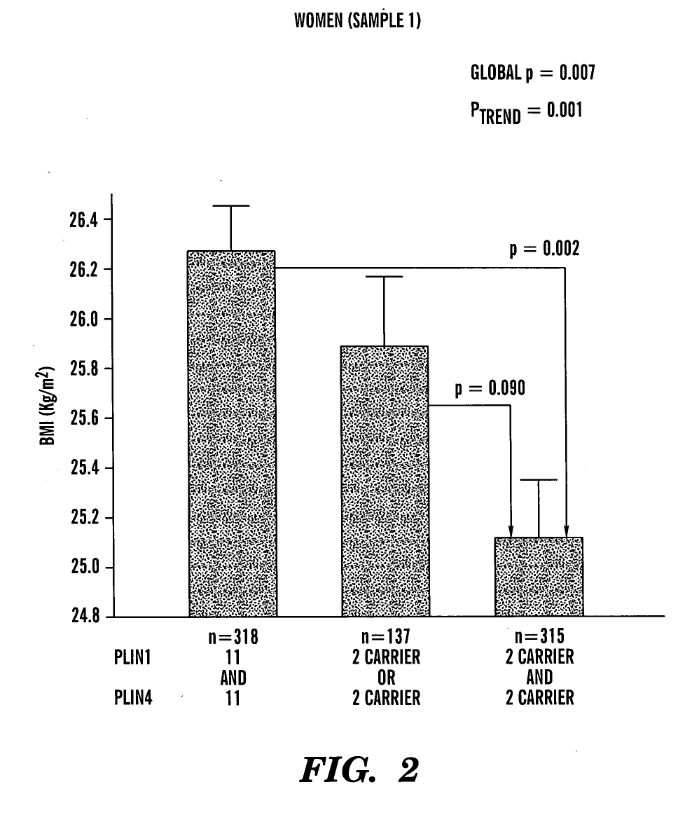

We also investigated the potential synergic associations between the PLIN1 and PLIN4 and relevant variables. Subjects from sample 1 were grouped into three categories: 1) homozygous for allele 1 at both PLIN1 and PLIN4 SNPs; 2) carriers of the 2 allele at either PLIN1 or PLIN4, and 3) carriers of the allele 2 at both PLIN1 and PLIN4. FIG. 2 shows age-adjusted means for BMI depending on the combined genotypes in women from sample 1. In addition, the model was adjusted for the PLIN5 and PLIN6 SNPs. The combined two-SNPs variable was significantly associated with BMI (p=0.007), with women homozygotes for the most common haplotype “11” showing higher BMI (26.3±0.3 Kg/m²; p=0.002) than women carrying at least one 2 allele 2 at both the PLIN1 and PLIN4 SNPs (25.1±0.3 Kg/m²). Carriers of at least one 2 allele at either the PLIN1 or PLIN4 SNPs had intermediate BMI phenotype. We also found statistically significant associations between the combined SNP variable and TAGs (p=0.020) and glucose (p=0.040), with homozygous for most common haplotype having the highest concentrations.

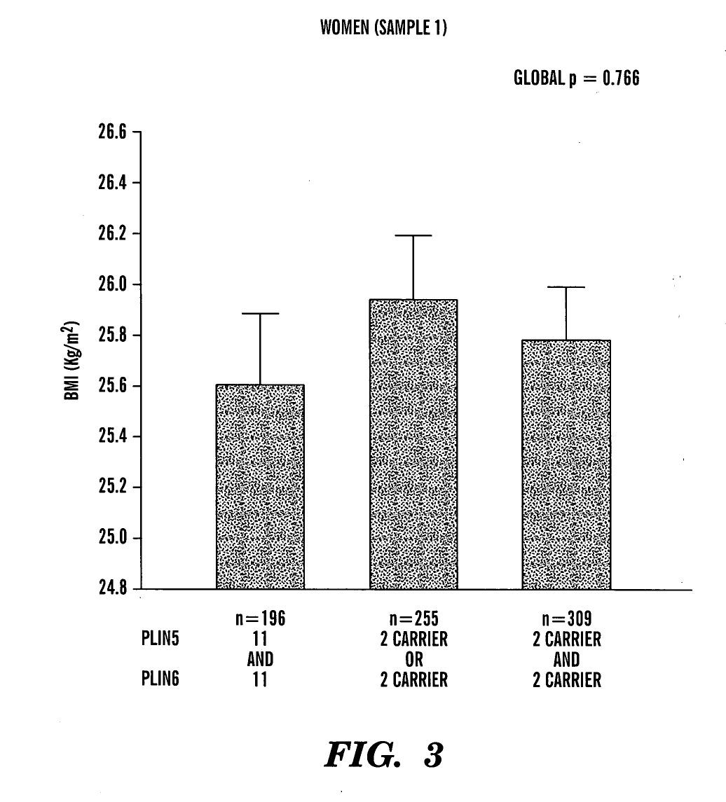

When this combined genotype analysis was performed on PLIN5 and PLIN6 polymorphism, after additional control for PLIN1 and PLIN4, no associations between this haplotype variable and any obesity-related parameters in men or women from sample 1 were detected. FIG. 3, shows age-adjusted means for BMI depending on the combined genotypes in women (sample 1). Although no significant, homozygous carriers of the most frequent haplotype had the lowest values of BMI as compared with the other haplotypes.

We carried out similar analyses using all four polymorphisms. For this purpose we considered four groups: 1) Subjects homozygotes for the most common alleles, haplotype “1111”; 2) Homozygotes for the most common allele at both PLIN1 and PLIN4 and carriers of the allele 2 at PLIN5 and PLIN6; 3) Carriers of the allele 2 at PLIN1 and PLIN4 and homozygotes for the most common allele at both PLIN5 and PLIN6; 4) Carriers of the 2 allele PLIN1, PLIN4 and PLIN5 and PLIN6. Subjects carrying any other genotype combination were not included in these analyses. In order to increase the statistical power, individuals from sample 1 and sample 2 were pooled and analyzed together. Table 7 shows age-adjusted means of weight and BMI in men and women depending on the combined genotype. In women, a highly statistically significant association between the combined genotype variable and weight and BMI was found, with carriers of the allele 2 at PLIN1 and PLIN4 locus and homozygotes for the most common allele at both PLIN5 and PLIN6 showing the lowest values. In men, we did not find any significant association between the genetic groups and BMI or body weight.

Risk of Obesity Associated with the PLIN Gene Variation

Finally to estimate the risk of obesity associated with the PLIN variants, subjects from sample 1 and sample 2 were pooled, and were subdivided according to categories of BMI: non-obese subjects (BMI<30 kg/m²), and obese (BMI≧30 kg/m²). In men, no significant differences in the prevalence of any PLIN polymorphism between obese and non obese were detected. However, in women, a lower prevalence of subjects carrying the allele 2 was detected for the PLIN1 polymorphism in obese as compared with non obese (50.2% vs. 60.4%; p=0.004). Since obese and non-obese differed in age, in the logistic regression model, the estimation of the risk (OR) was adjusted for age. After this adjustment, women carrying the allele 2 at the PLIN1 polymorphism, had a lower risk of obesity as compared with non-carriers: OR: 0.65; 95% CI, 0.48 to 0.88. Likewise, prevalence of women carrying the allele 2 at the PLIN4 polymorphism was lower in the obese group than in non obese (32.5% vs. 45.2%; p<0.001). After adjustment for age, the allele 2 at the PLIN4 locus was consistently associated with a lower risk of obesity in women, OR: 0.60; 95% CI, 0.44 to 0.83. Moreover, these estimations remained statistically significant after further adjustment for tobacco smoking, alcohol, consumption, physical activity, diabetes and education. In the two-polymorphisms combined genotype analysis and after adjustment for age, women carrying the allele 2 at both PLIN1 and PLIN4 SNPs, presented the lowest risk of obesity (OR: 0.56; 95% CI 0.39 to 0.79; p=0.001 as compared with the homozygotes for the most common alleles), whereas carriers of only one allele 2 at PLIN1 or at PLIN4 loci, showed non statistically significant differences in the risk of obesity as compared with the homozygotes for the most common alleles (OR: 0.95; 95% CI: 0.63 to 1.43). These results did not change after further adjustment for the PLIN5 and PLIN6 polymorphism. For PLIN5 and PLIN6 loci, neither in the single polymorphism analysis nor in the combined genotype analysis statistically significant associations with the risk of obesity were found.

Discussion

Studies using experimental models have demonstrated that perilipins play an important role in TAG storage in the adipocyte by regulating the rate of basal lipolysis and the hormonally stimulated lipolysis (7; 11,12). We have investigated the association of four common novel PLIN polymorphisms with measures of obesity, lipid metabolism and insulin sensitivity in a sample of Caucasian individuals and we have demonstrated for the first time that variations at the human PLIN locus are consistently associated with obesity-related variables, suggesting that perilipins may play a relevant role in human obesity, hypertriglyceridemia, and potentially on the development of the metabolic syndrome. Furthermore we have found that, in the general population, most of the associations were gender-specific affecting mostly women.

Association Between the PLIN Polymorphisms and Obesity-Related Phenotypes. Single Polymorphism Genotype Analysis.

In our analyses we have applied both, case-control and cross-sectional approaches to investigate the associations between the PLIN polymorphisms and obesity-related measures. In the case-control design including obese subjects from the general population and hospitalized obese patients, and after adjustment for age and other potential confounders, we have found a consistent and statistically significant lower risk of obesity in women carrying the allele 2 at the PLIN1 polymorphism. This association was also found with the allele 2 at the PLIN4 SNP but not with the PLIN5 or the PLIN6 polymorphisms. The strong linkage disequilibrium between PLIN1 and PLIN4 (D′>0.9), and their lesser linkage with the other 2 SNPs support these results. Moreover, the lower risk of obesity related to the less common alleles for the PLIN1 and the PLIN4 SNPs seen in women parallel findings on the perilipin null mouse linking the ablation of perilipin with a lean phenotype (11,13). In addition, inactivation of the PLIN gene also protected the Lepr(db/db) mice, a genetic model of obesity caused by leptin resistance, from developing obesity (13). The absence of significant associations in men from the general population highlights the importance of sex hormone factors in the regulation of body weight and fat distribution in humans.

In the sample from the general population, women carriers of the less common alleles for the PLIN1 and PLIN4 SNPs had statistically significant lower BMI than women homozygous for the most common allele. Moreover, we found that women carriers of the less common allele at the PLIN4 SNP had also significantly lower plasma glucose and TAGs concentrations. In addition, the PLIN4 polymorphism was also associated with decreased waist-to-hip ratio in women, suggesting a greater effect over the abdominal (visceral) fat depot. This finding is of particular importance, because abdominal (visceral) fat has been strongly associated with the metabolic syndrome: glucose intolerance, dyslipidemia, insulin resistance, hypertension, as well as cardiovascular disease and type 2 diabetes (19). Moreover, the same allele was also associated with lower fasting glucose levels. Along these lines, an interesting finding of our study is the consistent and statistically significant interaction between the PLIN polymorphisms and obesity in determining plasma glucose concentrations. In contrast, no significant associations were observed in men from the general population.

In obese women from sample 2, despite the consistent association between the allele 2 of the PLIN4 SNP with lower BMI, this allele was associated with higher plasma glucose concentrations. However, these results are in agreement with the observations of Tansey et al. (11) in perilipin knockout mouse and reconcile the findings of Martinez-Botas et al. (13). Fatty acid release from the adipose tissue are implicated in the development of type 2 diabetes, one might expect the Peri null mice to be susceptible to insulin resistance. Martinez-Botas et al. (13) failed to detect glucose intolerance in their Peri null animals, and more elaborated studies by Tansey et al. (11), replicated the findings of Martinez-Botas et al (13), in animals less than 30 g in weight. However, as the animals exceeded 30 g, significant glucose intolerance developed in the Peri null mice as compared with the wild-type. This is consistent with the notion that perilipin which protects against obesity may result in a more detrimental phenotype once the individual becomes obese. Moreover, although in men from the general population no effect of the PLIN alleles on plasma fasting glucose was found, in obese men the allele 2 was also associated with higher glucose concentrations, adding evidence to the effect of the obesity-interaction hypothesis. Another interesting finding related to the interaction between obesity and the PLIN SNPs relates to the association of the allele 2 at the PLIN4 locus with lower BMI in men from sample 2. These findings are consistent with the effect of this allele in women and raise the hypothesis that a higher adiposity or some undetected environmental factors special in obese men are needed to trigger the effects of the PLIN alleles.

The biological bases of these associations are unclear. None of the polymorphisms examined in our study appears to be functional. Both, the PLIN1 and the PLIN4 are intronic mutations. The PLIN5 is a silent mutation in exon 8, and the PLIN6 is in the untranslated region of exon 9. None of those mutations modify protein structure and, traditionally, they have not been considered to have regulatory functions. However, some evidence suggests that intronic polymorphisms might also regulate gene expression by affecting the binding of nuclear factors (20). The perilipins are the most abundant proteins coating the surfaces of lipid droplets in adipocytes (4-6). Their physiological relevance has become evident following recent reports showing that the PLIN null mouse had significantly decreased adipose stores and increased basal lipolysis in their isolated adipose cells as compared with the wild-type mouse (11,13). Based on these data, a possible explanation for our findings is that the PLIN1 and PLIN4 polymorphisms could be associated with lower expression of the PLIN gene or impaired perilipin activity. An alternative hypothesis is that these polymorphisms are directly involved, or in LD with mutations altering mRNA splicing. PLIN4, PLIN5 and PLIN6 are all close to the regions subject to alternative splicing (see FIG. 1). All the perilipins share an identical 22-kDa amino terminus with distinct carboxyl terminal sequences of varying lengths (21). The two major splice variants of the PLIN gene, perilipin A and perilipin B, showed distinct response to PKA activation and might exert different protection against lipolysis. The structural differences between these splice variants, especially the length of the C terminal tail affecting the wrapping of the droplet surface, may determine their functions.

The gender specific effects of the PLIN genotypes are consistent with the sex-specific differences in the development and distribution of adipose tissue, as well as the risks of obesity related diseases. The lipolytic capacity, one of the most important determinants of adipose tissue accumulation, was also shown to be gender dependent (22, 23). The present data do not allow for a determination of whether sex hormone could modify the effects of PLIN gene, and there is no data available at this time to explain the interaction between sex hormones and perilipin functions. We hypothesize that estrogen may amplify while testosterone may either have no effect on or minimize the protective effects of PLIN variants through unknown mechanisms that need elucidation.

Association Between the PLN Polymorphisms and Obesity-Related Phenotypes. Haplotype Analysis.

Our data show that the lowest risk of obesity was found in women carrying the allele 2 at both PLIN1 and PLIN4 SNPs, suggesting that these SNPs may work in an additive or synergic manner. Complex trait susceptibility may often be governed by the combined action of several different variants within a gene. Therefore, we propose that the biological effects of these markers are correlated but they are not associated with the same functional mutation.

Separately, both the PLIN5 and PLIN6 SNPs had no associations with BMI and other obesity related measures. However, haplotype analyses revealed a more interesting picture. We found that women carrying variant alleles of PLIN1 and PLIN4 but not PLIN5 and PLIN6 showed the lowest body weight and BMI (62.9 Kg and 24.8 kg/m²). Conversely, the presence of the variant alleles of PLIN5 and PLIN6 in the absence of the less common alleles for the PLIN1 and PLIN4 was associated with the highest body weight and BMI (72.2. Kg and 28.7 Kg/m²) a biologically significant difference of about 15% between the opposite haplotypes.

In conclusion, our study is the first one reporting associations between PLIN genotypes and obesity related measures in humans. This is consistent with recent findings from linkage analyses as well as with emerging data from animal models. A relevant issue that remains to be explored relates to the potential interactions between these SNPs and dietary factors. This is of relevance considering the relation between the expression of perilipin and the metabolism of fatty acids (24).

### Example 1 References

1. Hager J, Dina C, Francke S, Dubois S, Houari M, Vatin V, Vaillant E, Lorentz N, Basdevant A, Clement K, Guy-Grand B, and Froguel P (1998) A genome-wide scan for human obesity genes reveals a major susceptibility locus on chromosome 10. Nat. Genet., 20, 304-308.
2. Zamani, M., Pociot, F., Raeymaekers, P., Nerup, J., and Cassiman, J. J. (1996) Linkage of type I diabetes to 15q26 (IDDM3) in the Danish population. Hum. Genet., 98, 491-496.
3. Greenberg A S, Egan J J, Wek S A, Garty N B, Blanchette-Mackie E J, and Londos C (1991) Perilipin, a major hormonally regulated adipocyte-specific phosphoprotein associated with the periphery of lipid storage droplets. J. Biol. Chem., 266, 11341-11346.
4. Greenberg A S, Egan J J, Wek S A, Moos M C, Jr., Londos C, and Kimmel A R (1993) Isolation of cDNAs for perilipins A and B: sequence and expression of lipid droplet-associated proteins of adipocytes. Proc. Natl. Acad. Sci. U.S.A, 90, 12035-12039.
5. Nishiu J, Tanaka T, and Nakamura Y (1998) Isolation and chromosomal mapping of the human homolog of perilipin (PLIN), a rat adipose tissue-specific gene, by differential display method. Genomics, 48, 254-257.
6. Servetnick D A, Brasaemle D L, Gruia-Gray J, Kimmel A R, Wolff J, and Londos C (1995) Perilipins are associated with cholesteryl ester droplets in steroidogenic adrenal cortical and Leydig cells. J. Biol. Chem., 270, 16970-16973.
7. Brasaemle D L, Rubin B, Harten I A, Gruia-Gray J, Kimmel A R, and Londos C (2000) Perilipin A increases triacylglycerol storage by decreasing the rate of triacylglycerol hydrolysis. J. Biol. Chem., 275, 38486-38493.
8. Blanchette-Mackie E J, Dwyer N K, Barber T, Coxey R A, Takeda T, Rondinone C M, Theodorakis J L, Greenberg A S, and Londos C (1995) Perilipin is located on the surface layer of intracellular lipid droplets in adipocytes. J. Lipid Res., 36, 1211-1226.
9. Londos C, Brasaemle D L, Gruia-Gray J, Servetnick D A, Schultz C J, Levin D M, and Kimmel A R (1995) Perilipin: unique proteins associated with intracellular neutral lipid droplets in adipocytes and steroidogenic cells. Biochem. Soc. Trans., 23, 611-615.
10. Londos C, Gruia-Gray J, Brasaemle D L, Rondinone C M, Takeda T, Dwyer N K, Barber T, Kimmel A R, and Blanchette-Mackie E J (1996) Perilipin: possible roles in structure and metabolism of intracellular neutral lipids in adipocytes and steroidogenic cells. Int. J. Obes. Relat Metab Disord., 20 Suppl 3, S97-101.
11. Tansey J T, Sztalryd C, Gruia-Gray J, Roush, D L, Zee J V, Gavrilova O, Reitman M L, Deng C X, Li C, Kimmel A R, and Londos C (2001) Perilipin ablation results in a lean mouse with aberrant adipocyte lipolysis, enhanced leptin production, and resistance to diet-induced obesity. Proc. Natl. Acad. Sci. U.S.A, 98, 6494-6499.
12. Tansey J T, Huml A M, Vogt R, Davis K E, Jones J M, Fraser K A, Brasaemle D L, Kimmel A R, and Londos C (2003) Functional studies on native and mutated forms of perilipins: A role in protein kinase A-mediated lipolysis of triacylglycerols in CHO cells. J. Biol. Chem., 278:8401-8406.
13. Martinez-Botas J, Anderson J B, Tessier D, Lapillonne A, Chang B H, Quast M J, Gorenstein D, Chen K H, and Chan L (2000) Absence of perilipin results in leanness and reverses obesity in Lepr(db/db) mice. Nat. Genet., 26, 474-479.
14. Corella D, Guillen M, Saiz C, Portoles O, Sabater A, Cortina S, Folch J, Gonzalez J I, and Ordovas J M (2001) Environmental factors modulate the effect of the APOE genetic polymorphism on plasma lipid concentrations: ecogenetic studies in a Mediterranean Spanish population. Metabolism, 50, 936-944.
15. Corella D, Guillen M, Saiz C, Portoles 0, Sabater A, Folch J, and Ordovas J M (2002) Associations of LPL and APOC3 gene polymorphisms on plasma lipids in a Mediterranean population: interaction with tobacco smoking and the APOE locus. J. Lipid Res., 43, 416-427.
16. Sorli J V, Velert R, Guillen M, Portoles O, Ramirez J V, Iborra J, and Corella D (2002) [Effects of the apolipoprotein E polymorphism on plasma lipid levels and cardiovascular disease risk in a Mediterranean population]. Med. Clin. (Barc.), 118, 569-574.
17. Friedewald W T, Levy R I, and Fredrickson D S (1972) Estimation of the concentration of low-density lipoprotein cholesterol in plasma, without use of the preparative ultracentrifuge. Clin. Chem., 18, 499-502.
18. Antonarakis S E (1998) Recommendations for a nomenclature system for human gene mutations. Nomenclature Working Group. Hum. Mutat., 11, 1-3.
19. Gasteyger C, Tremblay A (2002) Metabolic impact of body fat distribution. J. Endocrinol. Invest, 25, 876-883.
20. Horikawa Y, Oda N, Cox N J, Li X, Orho-Melander M, Hara M, Hinokio Y, Lindner T H, Mashima H, Schwarz P E, Bosque-Plata L, HorikawaY, Oda Y, Yoshiuchi I, Colilla S, Polonsky K S, Wei S, Concannon P, Iwasaki N, Schulze J, Baier L J, Bogardus C, Groop L, Boerwinkle E, Hanis C L, and Bell G I (2000) Genetic variation in the gene encoding calpain-10 is associated with type 2 diabetes mellitus. Nat. Genet., 26, 163-175.
21. Lu X, Gruia-Gray J, Copeland N G, Gilbert D J, Jenkins N A, Londos C, and Kimmel A R (2001) The murine perilipin gene: the lipid droplet-associated perilipins derive from tissue-specific, mRNA splice variants and define a gene family of ancient origin. Mamm. Genome, 12, 741-749.
22. Lofgren P, Hoffstedt J, Ryden M, Thorne A, Holm C, Wahrenberg H, and Amer P (2002) Major gender differences in the lipolytic capacity of abdominal subcutaneous fat cells in obesity observed before and after long-term weight reduction. J. Clin. Endocrinol. Metab, 87, 764-771.
23. Kolehmainen M, Vidal H, Ohisalo J J, Pirinen E, Alhava E, and Uusitupa M I (2002) Hormone sensitive lipase expression and adipose tissue metabolism show gender difference in obese subjects after weight loss. Int. J. Obes. Relat Metab Disord., 26, 6-16.
24. Brasaemle, D. L., Barber, T., Kimmel,A. R., and Londos, C. (1997) Post-transnational regulation of perilipin expression. Stabilization by stored intracellular neutral lipids. J. Biol. Chem., 272, 9378-9387.

### Example II

### Gender Specific Association of a Perilipin (PLIN) Gene Haplotype with Obesity Risk in a White population from America

Materials and Methods

Subjects and Study Design

A total of 734 White subjects, 373 males (mean age 58.6 years) and 361 females (mean age 56.1 years) attending a residential lifestyle intervention program (The Pritikin Longevity Center, Santa Monica, Calif.) (19) were included in this study. In this population, current smoking was reported by 10.2%, and alcohol consumption (>1 drink/week) by 46.8% of the subjects. Medication use was as follows: 10.1% were taking hypoglycemic agents, 16.1% were on cholesterol-lowering drugs, 14.9% were on thyroid medication, and 35.7% of female subjects were on hormone replacement therapy. Due to limitations in DNA availability, genotypes were successfully obtained from 706 subjects for PLIN 6209T>C and 13041A>G, as well as from 705 subjects for PLIN 11482G>A and 14995A>T. Obesity was defined as BMI≧30 kg/m2. There were no significant differences in the anthropometrical and biochemical measures between the individuals with or without genotype information.

Biochemical Measurements

Fasting blood samples were drawn from all subjects at entry into the program (baseline). The blood samples were placed into tubes containing either SST clot-activating gel (Becton-Dickinson vacutainer system) for lipid and glucose measurements, or 0.1% EDTA for apolipoprotein measurements. The samples for lipid and glucose measurements were allowed to clot and serum was separated by centrifugation for 15 min at 2500 rpm. Total cholesterol (TC), high density lipoprotein cholesterol (HDL-C), triglyceride (TG), and glucose levels were measured by standardized automated enzymatic methods (Smith-Kline Beecham Laboratories), whilst low density lipoprotein cholesterol (LDL-C) was calculated as described previously (20).

DNA Isolation and Genotyping

Genomic DNA was isolated from whole blood using the QIA amp Blood Kit (Qiagen). Firstly, the DNA fragments containing target SNPs were amplified by multiplex polymerase chain reaction (PCR). The primers used are displayed in Table 1. PCR reactions were carried out in a 10 μl reaction volume containing 0.2 mmol/l of each dNTP, 0.2 μmol/l of each primer, 3.0 mmol/1 magnesium chloride, and 0.8 U of Qiagen Hotstar Taq polymerase. PCR cycling conditions were 95° C. for 10 min followed by 7 cycles of 95° C. for 30 seconds, 70° C. for 30 seconds, and 72° C. for 1 min, then followed by 41 cycles of 95° C. for 30 seconds, 65° C. for 30 seconds, and 72° C. for 1 min. A final extension phase of 5 min at 72° C. was included at the end of the protocol. The PCR products were incubated for 60 min at 37° C. with 2.5 U each of Exonuclease I (New England Biolabs., Inc. Beverly, Mass.) and Calf Intestinal Phospatase (New England Biolabs., Inc. Beverly, Mass.) to remove un-incorporated dNTPs and primers, and then followed by 15 min incubation at 75° C. to inactivate the enzymes. Single Nucleotide Extension was subsequently carried out using the ABI Prism SnaPshot system (Applied Biosystems, Foster City, Calif.). Probes used are presented in Table 1.

The reaction mixture for the extension reaction contained 1.5 μl of the Snapshot Ready Reaction Mastermix (Applied Biosystems, Foster City, Calif.), 1.0 μl of water, and 1.5 μl of multiplex PCR products and 1.0 μl of the probe mixture (2 μmol/l for each probe). The reaction conditions were 35 cycles of 96° C. for 30 seconds, 50° C. for 30 seconds, and 60° C. for 30 seconds. Products were incubated for 60 min at 37° C. with 3 U Calf Intestinal Phosphatase to remove un-incorporated dNTPs, followed by incubation for 15 min at 75° C. to inactivate the enzyme. Finally, genotyping was carried on an ABI Prism 3100 genetic analyzer (Applied Biosystems, Foster City, Calif.) using Genotyper version 3.7 (Applied Biosystems, Foster City, Calif.).

Statistical Analyses

Multivariate linear regression analysis was used to test the null hypotheses of no association between genetic variants and phenotypic outcomes adjusting for covariates (age, BMI, tobacco smoking, alcohol consumption, and medication status). ANCOVA (Tukey test) was employed to compare phenotypic outcomes between genotypic groups with multiple adjustments for covariates. An additive genetic model (grouping was based on the number of variant allele at each polymorphic site) was finally used according to the observed allelic effect. Interactions between gender and PLIN genotypes were tested by introduction of the corresponding product terms into the models. The SAS 8.0 statistical package was used to carry out hypothesis testing. A statistical P value less than 0.05 was considered as a significant boundary. Fasting glucose and triglycerides were logarithmically transformed to achieve a normal distribution before statistical testing. The THESIAS program was used to calculate allele frequency, to test pairwise linkage disequilibrium (LD), and to infer haplotypes. This computer program is based on the maximum likelihood model described by Tregouet et al (21). Haplotype association with obesity risk was examined with multiple adjustments for the covariates described above.

Results

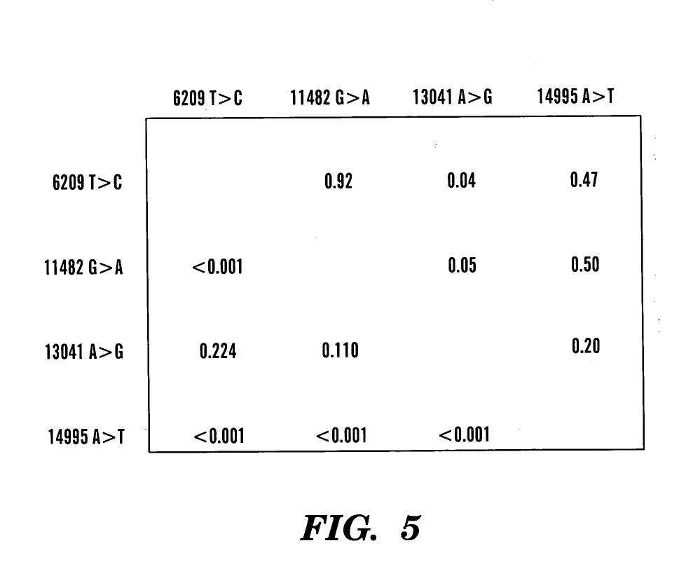

The identification of common polymorphisms at the PLIN locus was carried out by resequencing of conserved regions between humans and mice in 40 unrelated subjects and by searching one of the public SNP databases (http://www.ncbi.nlm.nih.gov/SNP/snp_ref.cgi?locusId=5346). Four common polymorphisms, PLIN 6209T>C, 11482G>A, 13041A>G, and 14995A>T, were identified and selected for this study. The numbering of these SNPs reflects their relative position to the A of the ATG of the initiator Methionine codon of PLIN, which was numbered as “+1” (at position 157157 on the reference sequence, accession number GI21431190). Genotype distributions did not deviate from Hardy-Weinberg expectations. Minor allele frequencies for the SNPs examined were 0.453 for 6209T, 0.299 for 11482A, 0.336 for 13041G, and 0.360 for 14995T. Examination of pair-wise linkage disequilibrium (LD) indicated that both PLIN 6209T>C and 11482G>A were in strong LD (D′=0.92, P<0.001). No significant LD were detected between these SNPs and the 13041 A>G SNP (D′=0.04, P=0.224 for 6209T>C/13041A>G pair, and D′=0.05, P=0.110 for 11482G>A/13041A>G pair). Finally, the PLIN 14995A>T showed different levels of LD as shown in FIG. 5.

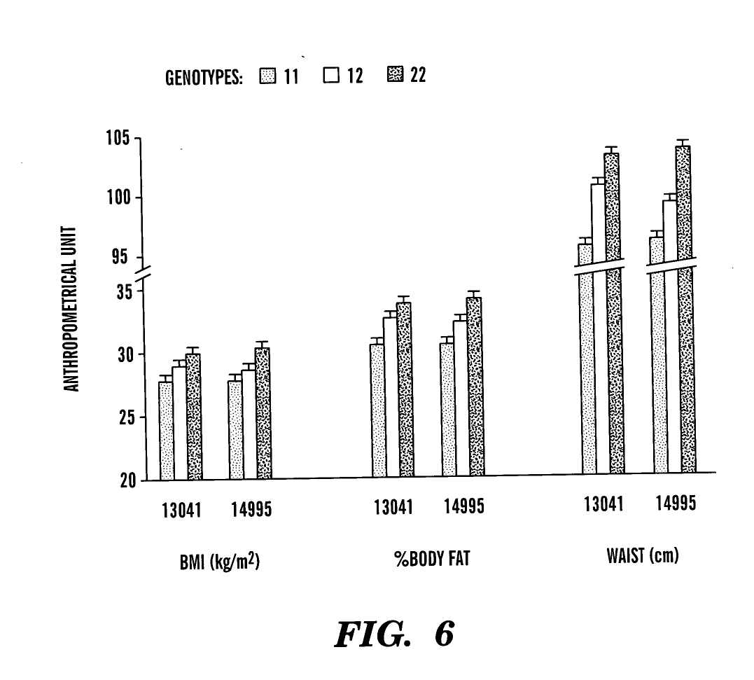

We found significant interactions between PLIN genotypes and gender for the outcome variables. Therefore, we carried out the analyses for men and women separately. First, we examined the allelic associations for each of the SNPs with body fat measures, including BMI, percent body fat, and waist circumference. In women, we found significant allelic differences in percent body fat and waist circumference. For PLIN 13041A>G, the mean percent body fat values for the AA, AG, and GG groups were 30.6%, 32.7%, and 33.3% respectively (P=0.0166). A similar association was observed for mean waist circumference: 95.1; 96.9; and 105.1 cm for AA, AG and GG subjects respectively (P=0.020). We observed similar associations for the PLIN 14995A>T SNP. Mean percent body fat in the AA, AT, and TT subjects was 30.5%, 32.5%, and 33.7% (P=0.0104); and mean waist circumference was 95.7, 98.9, and 102.6 cm respectively (P=0.0453). Subjects carrying the G/A and the G/G genotypes at the PLIN 13041A>G had BMI values 1.25 kg/m2 and 1.60 kg/m2 higher than AA subjects. Similarly, for the PLIN 14995A>T SNP, AT and TT subjects had 0.87 kg/m2 2.32 kg/m2 higher BMI than AA subjects (FIG. 6). No significant association was found between PLIN 6209T>C and PLIN 11482G>A genotypes and body fat measures in females. In men, there were no significantly genotype related differences for any of the variables examined (Data not shown)

We also examined the association between PLIN variations and the risk of obesity. We inferred haplotypes from the 4 SNPs and use these groups for further risk analyses. Haplotypes containing the minor alleles at SNPs 13041 or/and 14995 tended to had increased obesity risk, whereas haplotypes containing the minor alleles at the 6209 or/and 11482 tended to have decreased obesity risk in women. Among them, haplotype T/G/G/T was associated with the highest obesity risk (OR=2.09, 95% CI 0.83-5.23) and haplotype C/G/A/A was associated with the highest obesity protection (OR=0.58, 95% CI 0.25-1.34) after adjusting for covariates as previously described. (Table 2) However, none of these associations reached statistical significance due to limitations in sample size. To improve the study power, we also analyzed the haplotypic association based on either 6209T>C/1 1482G>A or 13041A>G/14995A>T haplotypes. We did not find any significant association between haplotypes inferred from 6209T>C/1 1482G>A in both men and women. When haplotypes inferred from 13041A>G/14995A>T were examined, both haplotype A/T (OR=1.76, 95% CI 1.07-2.90) and haplotype G/T (OR=1.73, 95% CI 1.06-2.82) were significantly associated with increased risk of obesity as compared with haplotype A/A in women (Table 8). We did not find significant association between 13041A>G/14995A>T haplotypes and the risk of obesity in men.

Because of the tight relationship between body fatness and the energy homeostasis, we then analyzed the association between PLIN genotypes and some metabolic measures related with energy homeostasis. In the female subjects, although associated with increased body fatness, PLIN 13041A>G and 14995A>T were not significantly associated with the metabolic measures examined. (Table 9) In contrast, PLIN 6209T>C and 11482G>A were associated with LDL-C level (P=0.007 for PLIN 6209T>C and P=0.028 for PLIN 11482G>A, Table 9). In addition, PLIN 11482G>A was also associated with TC level with marginal significance (P=0.068). Unlike the additive allele effects shown by PLIN 13041A>G/14995A>T on body fatness, only the carriers with homozygous variations of PLIN 6209T>C/11482G>A tend to have higher LDL-C or/and TC, while carriers of other genotypes had comparable levels in these measures. In the males, we found the study subjects who carried PLIN 13041G tend to had lower TC and LDL-C levels in comparison with those carrying wild type homozygotes. It was noticed such associations were all marginal (P=0.051 for TC and P=0.049 for LDL-C). In addition, a marginal association was also observed between PLIN 13041A>G and HDL-C level (P=0.047). However, it appeared the major difference of HDL-C level was between GA group and AA group. The genotypes of PLIN 6209T>C, 11482G>A, and 14995A>T were not associated with any metabolic measures examined in men (Table 10).

Discussion

First reported in the early 1990s, perilipin is emerging as a key regulator of lipolysis in adipocytes and body fat accumulation (14-17, 22-24). More recently, genetic variation at the PLIN locus was associated with decreased perilipin content and increased lipolytic activity in human adipocytes (18), supporting the role of PLIN as a candidate gene for obesity in the general population. In the present study, we have examined the association between variability at the PLIN locus and anthropometric and metabolic variables in a White population with elevated mean BMI. Among four common SNPs identified and genotyped in this population, we found that two SNPs (PLIN 13041A>G and 14995A>T) located in the 3′ untranslated region were significantly associated with increased percent body fat and waist circumference, as well as marginally associated with increased BMI in female subjects. Moreover, analyses of inferred haplotypes using the PLIN 13041A>G and 14995A>T SNPs demonstrated an increased risk of obesity for the A/T and G/T haplotypes. Conversely, in males, PLIN polymorphisms were not significantly associated with any of the measured parameters of body fatness.

Perilipins are expressed mostly in adipose cells and sterogenic cells. Because of their physical localization within fat depots, perilipins have been examined for their roles in regulating the mobilization of fat reserves and body fat accumulation and several in vitro studies have supported this notion (13,23,25). Further in vivo evidence for these roles came from the knockout mice models (15,16). Our current findings in relation to human PLIN gene variants are also consistent with the results derived from the experimental models, suggesting a conserved role of perilipin in lipolysis across different species.

Several perilipin isoforms have been identified resulting from alternative splicing (9,26) and these isoforms may be functionally different (24). Both, PLIN 13041A>G and 14995A>T are located in the 3′ untranslated region, where alternative splicing occurs during transcription. It is possible that these polymorphisms may alter the transcription product by affecting splicing. PLIN 13041A>G and 14995A>T are in significant LD with each other. Therefore, we postulate that the observed associations between these two polymorphisms and body fat measures may be pointing to the same causal mutation and, considering that the 14995T allele was consistently present in haplotypes associated with increased obesity risk, we hypothesize that this allele may be more closely associated with the causal mutation.

In our study, we examined several anthropometric measures (BMI, percent body fat and waist circumference). Although they are significantly correlated, these measurements are not identical in representing body fatness. Thus, BMI does not distinguish fat from lean mass. Moreover, these correlations are age dependent (27,28). On the other hand. waist circumference has been propose as a more precise measurement to identify those at higher risk for metabolic syndrome (29). Despite those differences, it is reassuring that we have found consistent associations between PLIN polymorphisms and several indices of obesity.

Measures of obesity are usually correlated with abnormalities in glucose and lipid metabolism. However, in our study we did not find significant associations between the PLIN 13041A>G and the 14995A>T SNPs and glucose or lipid-related measures. Similar findings have been observed in experimental models. Thus, the PLIN knockout mice appears to adapt to the constitutively activated lipolysis caused by PLIN gene ablation by activating mechanisms to dispose of these lipolytic products through upregulation of oxidative catabolic pathways and downregulation of lipid/sterol synthetic pathways (30). We suggest that such compensatory mechanisms may also take place when lipolysis is repressed.

The other two SNPs examined (PLIN 6209T>C and 11482G>A) were not associated with body adiposity in this study. PLIN 11482G>A was previously reported by Mottagui-Tabar et al. in association with decreased perilipin contents and increased lipolysis rate in obese women (18). Therefore, we expected PLIN 11482A would be associated with leanness phenotypes. Several reasons may account for the null association between this polymorphism and body fat measures in our study: First, our study population was more enriched in obese subjects than the general population (Mean BMI=29.6 kg/m2). It is possible these subjects were genetically predisposed to obesity due to the influence of other loci and that the expression of the protective effect of PLIN 11482A may be repressed under these conditions. Moreover, the PLIN 11482G>A polymorphism reported by Mottagui-Tabar's is an intronic SNP probably in LD with a functional mutation. As such, the association between PLIN 11482G>A and phenotypic variables could be affected by population specific genetic structure, in which the magnitude of pairwise LD between PLIN 11482G>A and the functional variation may be diminished in our population.

The finding that women who carried PLIN 11482AA genotype appeared to have higher TC and LDL-C was in line with Mottagui-Tabar's study in which AA genotype was associated with increased adipose lipolysis rate (18). The elevated fatty acid in circulation would increase their flux into the liver resulting in altered lipid metabolism and promote cholesterol production (31). Because PLIN 6209T>C and 111482G>A were in almost complete LD, we postulated the observed association between PLIN 6209 and LDL-C concentrations may have the same genetic basis that the PLIN 11482G>A SNP.

The PLIN locus was not associated with obesity related measures in male subjects. It has been proposed that men and women may have different sets of obesity susceptibility genes (7). In addition, twin studies suggest that obesity may be more inheritable in women than in men (32). However, larger studies are needed before we conclude that PLIN is not a candidate gene for obesity related phenotypes in men. The differential expression levels of perilipin in men and women (33) may account for their different sensitivity to the genetic effects of PLIN.

In summary, we found significant associations between two SNPs (PLIN 13041A>G and 14995A>T) at the 3′ untranslated region of the PLIN gene and obesity risk in White women. Carriers of the variant alleles at these two SNPs had increased mean body fat content, waist circumference, and BMI as compared with the carriers of the wild type genotypes. Conversely, no significant associations were found between PLIN polymorphisms and body fatness measures in men. Our findings support a significant role of PLIN as a candidate gene for obesity risk in women.

### Example II References

1. Bouchard C, Perusse L. Heredity and body fat. Annu Rev Nutr. 1988; 8:259-277.
2. Raj ala M W, Scherer P E. Minireview: The adipocyte—at the crossroads of energy homeostasis, inflammation, and atherosclerosis. Endocrinology 2003; 144:3765-3773.
3. Pereira A C, Floriano M S, Mota G F et al. Beta2 adrenoceptor functional gene variants, obesity, and blood pressure level interactions in the general population. Hypertension 2003; 42:685-692.
4. Coudreau S K, Tounian P, Bonhomme G et al. Role of the DGAT gene C79T single-nucleotide polymorphism in French obese subjects. Obes Res. 2003; 11: 1163-1167.
5. Doney A, Fischer B, Frew D et al. Haplotype analysis of the PPARgamma Pro 12Ala and C1431 T variants reveals opposing associations with body weight. BMC Genet. 2002; 3:21.
6. Lavebratt C, Ryden M, Schalling M et al. The hormone-sensitive lipase i6 gene polymorphism and body fat accumulation. Eur J Clin Invest. 2002; 32:938-942.
7. Amer P. Hunting for human obesity genes? Look in the adipose tissue! Int J Obes Relat Metab Disord 2000; 24 Suppl 4:S57-S62.
8. Amer P. Obesity—a genetic disease of adipose tissue? Br J Nutr. 2000; 83 Suppl 1: S9-16.
9. Greenberg A S, Egan J J, Wek S A et al. Isolation of cDNAs for perilipins A and B: sequence and expression of lipid droplet-associated proteins of adipocytes. Proc Natl Acad Sci. USA 1993; 90:12035-12039.
10. Nishiu J, Tanaka T, Nakamura Y. Isolation and chromosomal mapping of the human homolog of perilipin (PLIN), a rat adipose tissue-specific gene, by differential display method. Genomics 1998; 48:254-257.
11. Servetnick D A, Brasaemle D L, Gruia-Gray J et al. Perilipins are associated with cholesteryl ester droplets in steroidogenic adrenal cortical and Leydig cells. J Biol Chem. 1995; 270:16970-16973.
12. Brasaemle D L, Barber T, Wolins N E et al. Adipose differentiation-related protein is an ubiquitously expressed lipid storage droplet-associated protein. J Lipid Res. 1997; 38:2249-2263.
13. Souza S C, de Vargas L M, Yamamoto M T et al. Overexpression of perilipin A and B blocks the ability of tumor necrosis factor alpha to increase lipolysis in 3T3-L1 adipocytes. J Biol Chem. 1998; 273:24665-24669.
14. Brasaemle D L, Rubin B, Harten I A et al. Perilipin A increases triacylglycerol storage by decreasing the rate of triacylglycerol hydrolysis. J Biol Chem. 2000; 275:38486-38493.
15. Martinez-Botas J, Anderson J B, Tessier D et al. Absence of perilipin results in leanness and reverses obesity in Lepr(db/db) mice. Nat Genet. 2000; 26:474-479.
16. Tansey J T, Sztalryd C, Gruia-Gray J et al. Perilipin ablation results in a lean mouse with aberrant adipocyte lipolysis, enhanced leptin production, and resistance to diet-induced obesity. Proc Natl Acad Sci USA 2001; 98:6494-6499.
17. Kern P A, Di Gregorio G, Lu T et al. Perilipin expression in human adipose tissue is elevated with obesity. J Clin Endocrinol Metab. 2004; 89:1352-1358.
18. Mottagui-Tabar S, Ryden M, Lofgren P et al. Evidence for an important role of perilipin in the regulation of human adipocyte lipolysis. Diabetologia 2003; 46:789-797.
19. Barnard RJ. Effects of life-style modification on serum lipids. Arch Intern Med. 1991; 151:1389-1394.
20. Friedewald W T, Levy R I, Fredrickson DS. Estimation of the concentration of low-density lipoprotein cholesterol in plasma, without use of the preparative ultracentrifuge. Clin Chem. 1972; 18: 499-502.
21. Tregouet D A, Barbaux S, Escolano S et al. Specific haplotypes of the P-selectin gene are associated with myocardial infarction. Hum Mol Genet 2002; 11:2015-2023.
22. Londos C, Gruia-Gray J, Brasaemle D L et al. Perilipin: possible roles in structure and metabolism of intracellular neutral lipids in adipocytes and steroidogenic cells. Int J Obes Relat Metab Disord. 1996; 20 Suppl 3:S97-101.
23. Sztalryd C, Xu G, Dorward H et al. Perilipin A is essential for the translocation of hormone-sensitive lipase during lipolytic activation. J Cell Biol. 2003; 161:1093-1103.
24. Tansey J J, Huml A M, Vogt R et al. Functional studies on native and mutated forms of perilipins: A role in protein kinase A-mediated lipolysis of triacylglycerols in CHO cells. J Biol Chem. 2002.
25. Souza S C, Muliro K V, Liscum L et al. Modulation of hormone-sensitive lipase and protein kinase A-mediated lipolysis by perilipin A in an adenoviral reconstituted system. J Biol Chem. 2002; 277:8267-8272.
26. Lu X, Gruia-Gray J, Copeland N G et al. The murine perilipin gene: the lipid droplet-associated perilipins derive from tissue-specific, mRNA splice variants and define a gene family of ancient origin. Mamm Genome 2001; 12:741-749.
27. Prentice A M, Jebb S A. Beyond body mass index. Obes Rev. 2001; 2:141-147.
28. Allison D B, Saunders S E. Obesity in North America. An overview. Med Clin North Am. 2000; 84:305-32, v.
29 Visscher T L, Seidell J C, Molarius A et al. A comparison of body mass index, waist-hip ratio and waist circumference as predictors of all-cause mortality among the elderly: the Rotterdam study. Int J Obes Relat Metab Disord. 2001; 25:1730-1735.
30. Castro-Chavez F, Yechoor V K, Saha P K et al. Coordinated Upregulation of Oxidative Pathways and Downregulation of Lipid Biosynthesis Underlie Obesity Resistance in Perilipin Knockout Mice: A Microarray Gene Expression Profile. Diabetes 2003; 52:2666-2674.
31. Amer P. Insulin resistance in type 2 diabetes: role of fatty acids. Diabetes Metab Res Rev. 2002; 18 Suppl 2:S5-S9.
32. Herskind A M, McGue M, Sorensen T I et al. Sex and age specific assessment of genetic and environmental influences on body mass index in twins. Int J Obes Relat Metab Disord. 1996; 20:106-113.
33. Wang Y, Sullivan S, Trujillo M et al. Perilipin expression in human adipose tissues: effects of severe obesity, gender, and depot. Obes Res. 2003; 11:930-936.

### Example III

### Intragenic Linkage Disequilibrium Structure of the Human Perilipin Gene (PLIN) and Haplotype Association with Increased Obesity Risk in a Multi-Ethnic Asian Population

Materials and Methods

Subjects and Study Design

In total, 4,131 subjects who participated in the NHS 98 were included in this study. The NHS 98 was an initiative to determine the risk factors for the major non-communicable diseases in Singapore. The detailed methodology has been described previously (11). The procedures used in NHS 98 were based on the protocols and procedures recommended by the WHO for field surveys of diabetes and other non-communicable diseases and the WHO MONICA (Multi-national Monitoring of Trends and Determinants in Cardiovascular Disease) protocol for population surveys. In brief 11,200 individuals from addresses representing the house-type (a proxy for socio-economic status) distribution of the entire Singapore housing population were selected from the National Database on Dwellings. From these individuals, a random sample was selected by disproportionate stratified and systematic sampling. The Malays and Indians were over sampled, to ensure that prevalence estimates for these minority groups were reliable. A total of 4,723 subjects participated in the study, and, the ethnic composition was 64% Chinese, 21% Malays and 15% Asian Indians. Informed consent was obtained from all participants in the survey. The study was approved by the Ministry of Health in Singapore and the Ethics committee of the Singapore General Hospital.

Data on lifestyle factors were collected using an interviewer-administered questionnaire. Body fatness was evaluated using anthropometrical measures commonly employed for large scale epidemiological studies, including body weigh, body mass index (BMI), waist circumference, hip circumference, and waist/hip ratio (WHR). Briefly, body weight was measured in light indoor clothes without shoes using calibrated digital scales (SECA, Hamburg, Germany) with an accuracy of 0.1 kg. Body height was measured with the Frankfurt plane horizontal, to the nearest 0.1 cm without shoes using wall-mounted stadiometers. BMI was computed using body weight divided by the square of the body height (weight in kg/height in m2). Waist was measured to the nearest 0.1 cm, midway between the lower rib margin and the iliac-crest at the end of a gentle expiration. Measurements were taken directly on the skin. Hip circumference was measured to the nearest 0.1 cm over the great trochanters directly over the underwear (12). Obesity was defined dichotomously as BMI≧30 kg/m2, and, overweight was defined as 30 kg/m2>BMI≧25 kg/m2. There were 300 obese cases in total using the above criteria, while 1,333 subjects were categorized as overweight. No difference was found between subjects with and without genotyping on PLIN gene in the major anthropometrical and biochemical measures.

DNA Isolation and Genotyping

Genotyping was carried out using Single Nucleotide Extension. First, the DNA fragments encompassing five newly identified SNPs at PLIN locus were amplified by multiplex polymerase chain reaction (PCR). The SNPs were numbered (6209 T>C, 10171 A>T, 11482 G>A, 13041 A>G, 14995 A>T) according to their relative position to the A of the ATG of the initiator Methionine codon of PLIN, which was numbered as “+1” (at position 157157 on the reference sequence, accession number GI21431190). The primers used are presented in Table 1. PCR amplification was carried out in a 10111 reaction volume containing 0.2 mmol/l of each dNTP, 0.2 μmol/l of each primer, 3.0 mmol/1 magnesium chloride, and 0.8 U of Qiagen Hotstar Taq polymerase. PCR cycling conditions were 95° C. for 10 min followed by 7 cycles of 95° C. for 30 seconds, 70° C. for 30 seconds, and 72° C. for 1 min, then followed by 41 cycles of 95° C. for 30 seconds, 65° C. for 30 seconds, and 72° C. for 1 min. A final extension phase of 2 min at 72° C. was included at the end of the protocol. The PCR products were incubated for 60 min at 37° C. with 2.5 U each of Exonuclease I (New England Biolabs, Inc. Beverly, Mass.) and Calf Intestinal Phosphatase (New England Biolabs, Inc. Beverly, Mass.) to remove un-incorporated dNTPs and primers. This was followed by incubation for 15 min at 75° C. to inactivate the enzymes.

Subsequently, Single Nucleotide Extension was carried out using the ABI Prism SnaPshot multiplex system (Applied Biosystems, Foster City, Calif.). Probes used for Single Nucleotide Extension are listed in Table 1. The extension reaction was carried out using PCR thermocycler in a 5 μl reaction mixture containing 1.5 μl of the Snapshot Ready Reaction Mastermix (Applied Biosystems, Foster City, Calif.), 1.0 μl of water, and 1.5 μl of multiplex PCR products and 1.0 μl of the probe mixture (1.5 μmol/l for 6209C>T, 10171A>T, and 11482G>A; 2.0 μmol/l for 13041A>G and 14995A>T). The reaction conditions were 35 cycles of 96° C. for 30 seconds, 50° C. for 30 seconds, and 60° C. for 30 seconds. The reaction products were incubated for 60 min at 37° C. with 3 U Calf Intestinal Phosphatase to remove un-incorporated dNTPs, followed by incubation for 15 min at 75° C. to inactivate the enzyme. Genotyping was carried with the final products on an ABI Prism 3100 genetic analyzer (Applied Biosystems, Foster City, Calif.) using Genotyper version 3.7 (Applied Biosystems, Foster City, Calif.). The quality control for genotyping was established, and, the results were independently interpreted by two investigators.

Statistical Analyses

Arlequin (available at http://lgb.unige.ch/arlequin/) was used to estimate allele frequency, test the consistency of genotype frequencies at each SNP locus with Hardy-Weinberg equilibrium, and estimate pairwise LD between the SNPs examined. The statistical significance of LD between each pair of SNPs was tested using a likelihood-ratio test. Haplotypes were inferred using THESIAS program (Available at http://ecgene.net/genecanvas/modules/mydownloads/singlefile.php?cid=1&lid=1) that is designed for testing haplotype effects in unrelated subjects while adjusting for covariates. This computer program is based on the maximum likelihood model described by Tregouet et al (13). SAS (Windows version 8.0) was used to analyze individual associations, and statistical significance was defined at the 5% level. Differences in the prevalence of PLIN genotypes between obese cases and non-obese controls were analyzed by x² analysis. Odds ratios (OR) with 95% confidence intervals (CI) were used to estimate the relative risk of obesity. Multivariable logistic regression analysis was used to control for potential covariates for obesity (age, gender, cigarette smoking, alcohol consumption, exercise, and diabetes status). Interaction between genetic effect and gender was tested by introducing the corresponding product term into the model. A general inheritance model (subjects were groups according to the genotypes of each SNP) was first employed for examining the allele effect, and, appropriate inheritance models (dominant, recessive, or additive) were finally used based on observed allelic effects.

Results

Five common diallelic polymorphisms (6209T>C, 10171A>T, 11482G>A, 13041A>G, and 14995A>T) were selected and genotyped in the Singapore NHS98 population. These SNPs are located at intron 2 (6209), intron 5 (10171), intron 6 (11482), exon 8 (13041) and exon 9 (14995) respectively. Genotypic information for the five PLIN polymorphisms was obtained from 4,131 study subjects. The characteristics of the genotyped participants are shown in Table 11. Chinese represented 67.28%, 18.16% were Malays, and 14.56% were Indians. Overall, Indians were older and Chinese were younger. In men, Malays and Indians had comparable mean BMI, which was ˜1.0 kg/m2 higher than that in Chinese. In women, Malays had the highest BMI (26.3±5.6 kg/m2), followed by Indian (25.6±5.0 kg/m2) and Chinese (22.1±3.6 kg/m2). For both men and women, obesity (BMI≧30 kg/m2) and overweight (BMI≧25 kg/m2) were most prevalent in Malays, followed by Indians. The prevalence of obesity and overweight in these two ethnic groups were much higher than that in Chinese. Indian men and women had the highest rates of diabetes mellitus (18.2% for men and 17.4% for women), higher than those observed in Malays (10.9% for men and 14.8% for women) whereas in Chinese these numbers were much lower at 7.2% for men and 6.6% for women. Malays had highest proportion of current smoker while alcohol was most frequently consumed among Chinese.

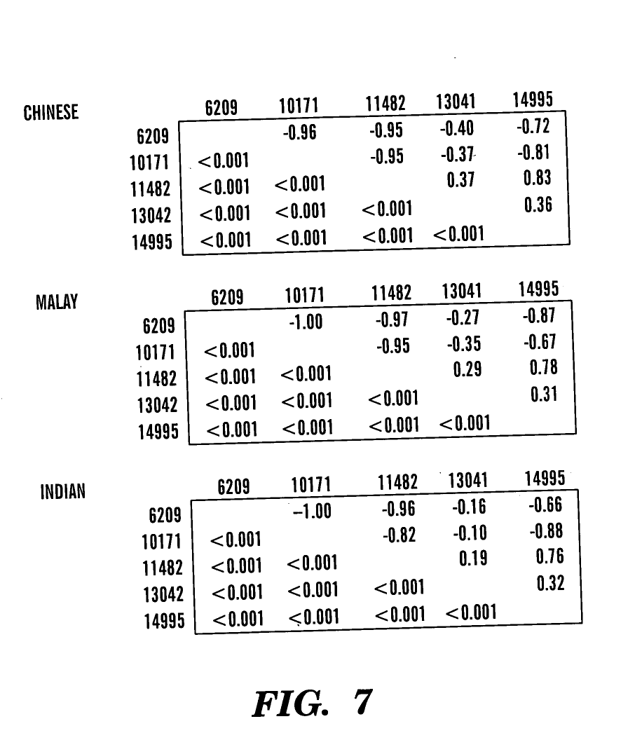

Among the three ethnic groups, the frequencies for the minor alleles ranged from 0.320 to 0.462 for PLIN 6209C>T, from 0.135 to 0.255 for PLIN10171A>T, from 0.326 to 0.439 for PLIN 11482G>A, from 0.296 to 0.471 for PLIN 13041A>G, and from 0.361 to 0.444 for PLIN 14995A>T. The observed and expected genotype frequencies were consistent with Hardy-Weinberg equilibrium for all polymorphisms in the three ethnic groups. Chi-square test for homogeneity showed that there were no significant differences in genotypic/allelic distribution between men and women for any of the five SNPs examined. Conversely, we observed significant between-ethnic differences in the genotype distribution at each polymorphic site. Significant non-random allelic associations were found between each pair of SNPs, as indicated by D′ for the pair-wise LD in FIG. 7. It appears that the LD structure within PLIN was not uniform. Both the PLIN 6209C>T and 10171 A>T SNPs were in negative LD with all other SNPs, whereas the PLIN 11482G>A, 13041A>G and 14995A>T SNPs were in positive LD with each other in three ethnic groups. Among the positive associations, the strongest LD was found between PLIN 11482G>A and 14995A>T, with D′ ranging from 0.76 to 0.83 among the three ethnic groups.

We examined the potential association between inferred PLIN haplotypes and obesity (Defined as BMI≧30 kg/m2) risk in the three ethnic groups. We have used THESIAS based on maximum likelihood algorithm for haplotype reconstruction (13). We did not detect significant gene-gender interactions. Therefore, men and women were analyzed together. Using five SNPs, we inferred 24, 18, and 18 haplotypes for Chinese, Malay, and Indians, respectively. We then examined the association between the common haplotypes (with frequencies higher than ˜5%) and obesity risk (Table 12). In Malays, we found that haplotypes 11222 (OR=1.64, 95% CI 1.08-2.48) and 11212 (OR=1.65, 95% CI 1.11-2.46) were significantly associated with increased risk of obesity compared with the most prevalent haplotype 21111. Haplotype 11212 was also found significantly associated with obesity risk in Indians (OR=1.94, 95% CI 1.06-3.53). Conversely, haplotype 12111, was associated with decreased risk of obesity compared with haplotype 21111 reaching marginal significance in Indians (OR=0.30, 95% CI 0.09-1.06). Likewise, this haplotype was also associated with slightly decreased obesity risk in Malays. Adjustment for relevant covariates (age, sex, smoking, alcohol consumption, exercise, and diabetes status) did not change the significance of observed association in Malays but slightly reduced the significance in Indians. We did not find significant associations between PLIN haplotypes and obesity risk in Chinese.

We also examined haplotype associations using a subgroup of SNPs (PLIN 11482, 13041, and 14995), which are in positive LD with each other. With these three SNPs, we inferred eight haplotypes within each ethnic group. Tests for the association between the individual haplotypes (Frequency greater than ˜5%) and obesity risk indicated that, in Malays, haplotype 212, 222, and 121 were significantly associated with increased odds for obesity as compared with the most common haplotype 111 (OR=2.12, 95% CI 1.36-3.32 for 212, OR=2.02, 95% CI 1.36-3.01 for 222, and OR=1.89, 95% CI 1.05-3.41 for 121). In Indians, haplotype 212 was significantly associated with increased odds for obesity as compared to haplotype 111 (OR=2.39, 95% CI 1.26-4.50). Haplotype 122 was also associated with increased obesity risk with marginal significance. Adjustment for the major obesity risk factors (age, sex, cigarette smoking, alcohol consumption, exercise, and diabetes status) did not change the observed associations except that the association with haplotype 121 in Malays became marginally significant. (Table 13 and Table 14).

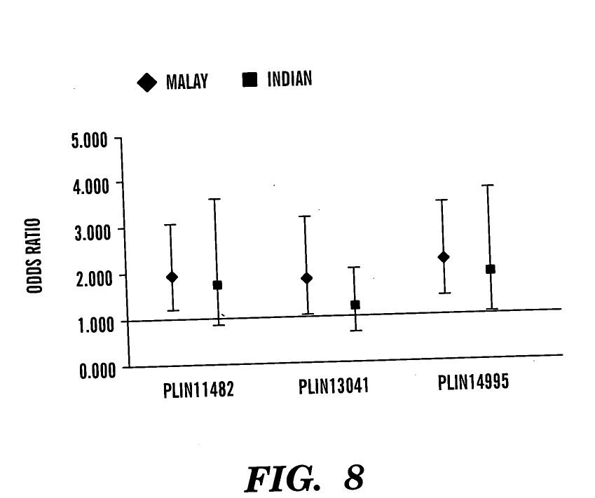

In addition, we examined each individual SNP for its association with the risk of obesity. No significant association was found with PLIN 6209C>T and 11482G>A. Homozygosity for the T allele at PLIN 14995A>T was significantly associated with increased odds of obesity as compared with other genotypes in both Malays and Indians (Multivariate OR=2.28, 95% CI 1.45-3.57 for Malays, and multivariate OR=2.04, 95% CI 1.08-3.84 for Indians). Homozygosity for the rare allele of either the PLIN 11482G>A or 13041 A>G was also found associated with increased odds of obesity in Indians and Malays, but only in the later group reached statistical significance (Multivariate OR=1.94, 95% CI 1.22-3.08 for PLIN 11482G>A, and multivariate OR=1.87, 95% CI 1.08-3.25 for PLIN 13041A>G) (See FIG. 8). No significant associations were found between these polymorphisms and obesity risk in Chinese.

Discussion

In this study, we have investigated the associations between PLIN gene variants and the risk of obesity in 4,131 subjects with different ethnic backgrounds using SNP and haplotype-based analyses. We genotyped five biallelic polymorphisms at the PLIN locus, (PLIN 6209C>T, 10171A>T, 11482G>A, 13041A>G, and 14995A>T), a candidate gene for obesity, in an Asian population including three ethnic groups (Chinese, Malays and Indians). By examining the association of inferred haplotypes with the risk of obesity, we demonstrated that the PLIN 11212 haplotype was significantly associated with increased risk for obesity in Malays and Indians. Additional haplotype analysis using three of the SNPs that were in positive linkage disequilibrium (11482G>A, 13041A>G, and 14995A>T) indicated that haplotypes 212 and 222 were associated with increased obesity risk in Malays, and haplotype 212 was significantly associated with increased obesity risk in Indians after covariate adjustment. Finally, individual SNPanalysis revealed that the PLIN 14995A>T was significantly associated with obesity risk in both Malays and Indians.

Our findings provide strong support for the consideration of PLIN as a candidate gene for obesity risk in humans. (Refer to http://obesitygene.pbrc.edu/) Perilipin is the predominant lipid droplet associated protein in adipocytes (2,3,14). It has been found that perilipin may play important roles in regulating PKA-mediated intracellular lipolysis in adipocytes, and, influencing the turn-over of stored TAGs (4,5,15). In vivo experimental models have demonstrated that the product of the PLIN gene plays a critical role in determining body fat composition (6,7). In humans, the abundance of perilipin in adipose tissue was also associated with lipolysis rate, and one of its genetic variants may influence both perilipin content and lipolysis rate (8).

Our data show consistent associations between PLIN haplotypes and obesity risk in two of the three ethnics examined. Haplotype 11212 was consistently associated with increased obesity risk in Malays and Indians, suggesting that this haplotype may contain the functional mutation. Moreover, haplotype analyses using SNPs at sites 11482, 13041, and 14995 increased the magnitude and statistical significance of the association. Haplotype 212 (at 11482, 13041, and 14995) was associated with increased obesity risk as compared with the wild type haplotype (111) across Malays and Indians, after adjusting for relevant covariates. Given the consistent association with increased obesity risk in both ethnic groups, we hypothesize that haplotype 212, derived from the 11482G>A, 13041A>G, and 14995A>T SNPs, more likely harbors or cosegregates with the functional mutation.

The results from analyzing individual SNPs suggested that PLIN 14995A>T was the most significant single genetic contributor for the observed haplotype association with obesity. This polymorphism was consistently associated with obesity risk in both Malays and Indians and carried the highest odds ratios. Although the other two SNPs, PLIN 11482G>A and 13041A>G, were also found associated with increased risk of obesity, the lesser magnitude of the findings and the fact that were restricted only to one of the ethnic groups suggest that their association may be due to their LD with the PLIN 14995A>T SNP.

We did not find significant association between PLIN variation and obesity risk in Chinese. Some researchers have proposed that a lower cutoff should be applied to define obesity in Asians (16,17). However, using lower cutoffs (27 kg/m2 and 25 kg/m2) in our analysis did not change the magnitude of the findings (data not shown). Alternatively, we postulate that differential penetrance of the genetic effects may be the underlying reason accounting for the observed discrepancy between Chinese and other two ethnic groups in terms of the relation between PLIN and obesity. In Singapore, Malays and Indians have comparable mean BMIs, which are significantly higher than the mean BMI in Chinese, despite living in a similar environment, suggesting that Chinese may have a lower genetic predisposition to obesity.

The PLIN 13041A>G and PLIN 14995A>T SNPs are located in the region where alternative splicing occurs during PLIN transcription resulting in several perilipin isoforms (18). Recent data showed that perilipin isoforms might function with different efficiency in protecting the storage fat from the PKA-mediated lipolysis (19). Therefore, without wishing to be bound by theory, it is possible that the genetic effect underlying the associations with PLIN 13041A>G and PLIN 14995A>T may be through affecting splicing and the expression of different perilipin isoforms. It is also possible that the PLIN 11482G>A just represents a genetic marker, rather than a functional mutation, in these associations. We have noted important differences in LD structure between Asian and Caucasian populations for the PLIN gene (data not shown) and we argue that the different intragenic LD structure between different ethnic groups may drive to different associations in various ethnic groups. Such differences in LD structure could explain the discrepancy between our findings and those of an earlier study. Mottagui-Tabar et al. recently reported that the A allele at the PLIN 11482G>A SNP was associated with enhanced basal and noradrenaline induced lipolysis. Moreover, the same allele was associated with lower perilipin content in obese women (8). According to this finding, and opposite to our observations, a negative association would be expected between PLIN 11482AA genotype and body fat. However, in the study by Mottagui-Tabar et al., the subjects were Caucasian females. Ethnic differences in LD structure could also explain the lack of association between genetic variants at this locus and obesity in Chinese.

In summary, we found a consistent association between PLIN haplotypes and increased obesity risk in Singaporean Malays and Indians. A common risk haplotype may be shared by Malays and Indians predisposing these ethnic groups to obesity. Single SNP analysis suggests that the PLIN 14995A>T might be the more relevant genetic marker for the observed haplotype associations.

### Example III References

1. Greenberg A S, Egan J J, Wek S A et al. Perilipin, a major hormonally regulated adipocyte-specific phosphoprotein associated with the periphery of lipid storage droplets. J Biol Chem 1991; 266: 11341-11346.
2. Greenberg A S, Egan J J, Wek S A et al. Isolation of cDNAs for perilipins A and B: sequence and expression of lipid droplet-associated proteins of adipocytes. Proc Natl Acad Sci USA 1993; 90: 12035-12039.
3. Servetnick D A, Brasaemle D L, Gruia-Gray J et al. Perilipins are associated with cholesteryl ester droplets in steroidogenic adrenal cortical and Leydig cells. J Biol Chem 1995; 270: 16970-16973.
4. Brasaemle D L, Rubin B, Harten I A et al. Perilipin A increases triacylglycerol storage by decreasing the rate of triacylglycerol hydrolysis. J Biol Chem 2000; 275: 38486-38493.
5. Souza S C, de Vargas L M, Yamamoto M T et al. Overexpression of perilipin A and B blocks the ability of tumor necrosis factor alpha to increase lipolysis in 3T3-L1 adipocytes. J Biol Chem 1998; 273: 24665-24669.
6. Martinez-Botas J, Anderson J B, Tessier D et al. Absence of perilipin results in leanness and reverses obesity in Lepr (db/db) mice. Nat Genet 2000; 26: 474-479.
7. Tansey J T, Sztalryd C, Gruia-Gray J et al. Perilipin ablation results in a lean mouse with aberrant adipocyte lipolysis, enhanced leptin production, and resistance to diet-induced obesity. Proc Natl Acad Sci USA 2001; 98: 6494-6499.
8. Mottagui-Tabar S, Ryden M, Lofgren P et al. Evidence for an important role of perilipin in the regulation of human adipocyte lipolysis. Diabetologia 2003; 46: 789-797.
9. Poulsen P, Vaag A, Kyvik K, Beck-Nielsen H. Genetic versus environmental aetiology of the metabolic syndrome among male and female twins. Diabetologia 2001; 44:537-43.
10. Hughes K, Aw T C, Kuperan P et al. Central obesity, insulin resistance, syndrome X, lipoprotein (a), and cardiovascular risk in Indians, Malays, and Chinese in Singapore. J Epidemiol Community Health 1997; 51: 394-399.
11. Cutter J, Tan B Y, Chew S K. Levels of cardiovascular disease risk factors in Singapore following a national intervention programme. Bull World Health Organ 2001; 79: 908-915.
12. Deurenberg-Yap M, Li T, Tan W L et al. Can dietary factors explain differences in serum cholesterol profiles among different ethnic groups (Chinese, Malays and Indians) in Singapore? Asia Pac J Clin Nutr 2001; 10: 39-45.
13. Tregouet D A, Barbaux S, Escolano S et al. Specific haplotypes of the P-selectin gene are associated with myocardial infarction. Hum Mol Genet 2002; 11: 2015-2023.
14. Nishiu J, Tanaka T, Nakamura Y. Isolation and chromosomal mapping of the human homolog of perilipin (PLIN), a rat adipose tissue-specific gene, by differential display method. Genomics 1998; 48: 254-257.
15. Sztalryd C, Xu G, Dorward H et al. Perilipin A is essential for the translocation of hormone-sensitive lipase during lipolytic activation. J Cell Biol 2003; 161: 1093-1103.
16. Chang C J, Wu C H, Chang C S et al. Low body mass index but high percent body fat in Taiwanese subjects: implications of obesity cutoffs. Int J Obes Relat Metab Disord 2003; 27: 253-259.
17. WHO expert consultation. Appropriate body-mass index for Asian populations and its implications for policy and intervention strategies, The Lancet 2004; 363: 157-163.
18. Lu X, Gruia-Gray J, Copeland N G et al. The murine perilipin gene: the lipid droplet-associated perilipins derive from tissue-specific, mRNA splice variants and define a gene family of ancient origin. Mamm Genome 2001; 12: 741-749.
19. Tansey J J, Huml A M, Vogt R et al. Functional studies on native and mutated forms of perilipins: A role in protein kinase A-mediated lipolysis of triacylglycerols in CHO cells. J Biol Chem 2002.

The references cited herein and throughout the specification are herein incorporated by reference in their entirety.

TABLE 1

| | |
|---|---|
| SNPs | Primers and probes |

| | |
|---|---|
| PLIN1 (6209¹ T > C) | Forward: CTCTGTTCTCCAGGGACCAAGTCAGAT |
| | (SEQ ID NO.: 1) |
| dbSNP rs#2289487² | Reverse: CCTACACTCTGGGGATGCGGAGAT |
| | (SEQ ID NO.: 2) |
| Intron 2 | Probe: |
| Contig. | GACTGACTGACTGACTGACTGACCCCACTGCCTAGAA |
| Position: 150949³ | (SEQ ID NO.: 3) |
| PLIN2 (N.D.)⁴ | Forward: GAGGGAGAAGAGAGGTGTGAGAGGGA |
| | (SEQ ID NO.: 4) |
| Intron 3 | Reverse: CATCTGGGCTCTCTGCTGCTTGAG |
| | (SEQ ID NO.: 5) |
| dbSNP rs#1561726 | Probe: |
| Contig. | GACTGACTGACTGACTGACTGACTGACTGTG |
| Position: 149309 | CCCCCGGAGAG |
| | (SEQ ID NO.: 6) |
| PLIN3 (10171 A > T⁵) | Forward: TTGGCCTTGGGAGACTTCTGGG |
| | (SEQ ID NO.: 7) |
| dbSNP rs#2304794 | Reverse: TTGTCACACACACTGCCTGGGAAT |
| | (SEQ ID NO.: 8) |
| Intron 5 | Probe: |
| Contig. | GACTGACTGACTGACTGACTGACTGACTGACT |
| Position: 146987 | GCAGGAGGTGGCTCA |
| | (SEQ ID NO.: 9) |
| PLIN4 (11482 G > A) | Forward: AAGTGTTGCCCCTGCAGGAAT |
| | (SEQ ID NO.: 10) |
| dbSNP rs#894160 | Reverse: GAGTGGAACTGCTGGGCCATA |
| | (SEQ ID NO.: 11) |
| Intron 6 | Probe: |
| Contig. | GACTGACTGACTGACTGACTGACTGACTGACTGA |
| Positoin: 145676 | CTTGTGGGGCTCCCTAGA |
| | (SEQ ID NO.: 12) |
| PLIN5 (13041 A > G) | Forward: CTCACCGGCACGTAATGCAC |
| | (SEQ ID NO.: 13) |
| dbSNP rs#2304795 | Reverse: CCCTCCAGACCACCATCTCG |
| | (SEQ ID NO.: 14) |
| Exon 8 (synonymous) | Probe: |
| Contig. | GACTGACTGACTGACTGACTGACTGACTGACTGAC |
| Position: 144116 | TGACCTTGGTTGAGGAGACAGC |
| | (SEQ ID NO.: 15) |
| PLIN6 (14995 A > T) | Forward: AAGCAGCTGGCTCTACAAAGCA |
| | (SEQ ID NO.: 16) |
| dbSNP rs#1052700 | Reverse: AGCATCCTTTGGGGCTTCA |
| | (SEQ ID NO.: 17) |
| Exon 9 (untranslated | Probe: |
| region) | GACTGACTGACTGACTGACTGACTGACTGACTGACTGA |
| Contig. | CTGACTGACTGCCTGCTGGGAGCCT |
| Position: 142163 | (SEQ ID NO.: 18) |

¹The coding number is the number of bases from the variants and the A of ATG of the initiator Methionine codon which is denoted nucleotide +1.
²Refer to “http://www.ncbi.nlm.nih.gov/SNP/snp_ref.cgi?locusId=5346”.
³The genomic position in reference sequence (GI21431190).
⁴Not detected;
⁵Observed less common allele frequency is less than 2%.

TABLE 2
Demographic, biochemical and life-style characteristics of the study subjects depending on the sample
selection: sample 1 (population-based), and sample 2 (Hospital-based)

| | Sample 1 | | | | Sample 2 | | | |
|---|---|---|---|---|---|---|---|---|
| | Men | | Women | | Men | | Women | |
| | (n = 788) | | (n = 801) | | (n = 29) | | (n = 128) | |
| | Mean (SD) | | Mean (SD) | | Mean (SD) | | Mean (SD) | |
| Age (years) | 40.6 | (11.6) | 42.4 | (14.8)* | 47.5 | (14.1) | 47.4 | (13.6) |
| Body weight (kg) | 78.9 | (11.1) | 64.4 | (12.7)* | 125.2 | (29.5) | 106.8 | (19.1)* |
| Body height (m) | 1.73 | (0.06) | 1.59 | (0.06)* | 1.74 | (0.07) | 1.58 | (0.05)* |
| Body mass index (kg/m²) | 26.4 | (3.5) | 25.7 | (5.4)* | 40.9 | (8.9) | 42.7 | (8.2) |
| Waist (cm) | 95.6 | (11.1) | 88.3 | (15.4)* | 128.2 | (18.1) | 120.0 | (16.7) |
| Hip (cm) | 100.8 | (9.9) | 102.0 | (13.0) | 126.0 | (21.3) | 132.4 | (11.6) |
| Waist-to-hip ratio | 0.95 | (0.07) | 0.86 | (0.07)* | 1.02 | (0.12) | 0.91 | (0.08)* |
| Fasting glucose (mg/dL) | 92.6 | (24.4) | 96.1 | (20.3)* | 126.2 | (54.2) | 120.4 | (16.7) |
| Triglycerides (mg/dL) | 129.5 | (80.4) | 94.5 | (56.6)* | 147.7 | (72.8) | 148.2 | (83.8) |
| Total-C (mg/dL) | 206.4 | (38.8) | 201.4 | (38.4)* | 187.1 | (30.4) | 204.0 | (41.9) |
| LDL-C (mg/dL) | 134.7 | (34.8) | 128.1 | (33.2)* | 112.7 | (30.3) | 125.2 | (33.7) |
| HDL-C (mg/dL) | 46.6 | (9.8) | 54.9 | (11.5)* | 44.7 | (13.1) | 50.5 | (13.9)* |
| Systolic blood pressure (mmHg) | 124.7 | (16.1) | 123.2 | (21.6) | 139.0 | (15.0) | 136.7 | (15.6) |
| Diastolic blood pressure (mmHg) | 75.6 | (10.5) | 74.6 | (12.5) | 83.7 | (11.6) | 84.9 | (11.1) |

| | | | | |
|---|---|---|---|---|
| Obesity (BMI >= 30 kg/m²) (%) | 15.0 | 20.3* | 100.0 | 100.0 |
| Overweight (BMI >= 25 kg/m²) (%) | 61.7 | 46.6* | 100.0 | 100.0 |
| BMI > 35 kg/m² (%) | 1.6 | 6.9 | 79.3 | 89.1 |
| Current smokers (%) | 39.5 | 33.2* | 35.7 | 26.7* |
| Alcohol users (%) | 90.6 | 56.8* | 66.7 | 30.8* |
| Physical exercise (%) | | | | |
| Sedentary | 36.3 | 58.4* | 96.0 | 74.8* |
| Active | 63.7 | 41.6 | 4.0 | 25.2 |
| Education (%) | | | | |
| Primary | 43.7 | 47.1* | 66.7 | 75.2 |
| Secondary | 32.3 | 22.3 | 18.5 | 16.5 |
| University (I and II) | 24.0 | 30.5 | 14.8 | 8.3 |
| Type 2 diabetes (%) | 3.8 | 4.3 | 14.3 | 21.5 |
| Taking lipid lowering drugs (%) | 5.7 | 8.1 | 14.3 | 21.5 |

SD: Standard deviation.
Total-C: Total cholesterol.
LDL-C: low-density lipoprotein cholesterol.
HLD-C: high-density lipoprotein cholesterol.
*P value <0.05 in the comparison between men and women. Student's t test for comparison of means, and Chi square tests for percentages.
University I: 3 years.
University II: 5 years or more

TABLE 3
Genotype distribution, allele frequencies and linkage disequilibrium of the polymorphic gene
variants at the PLIN locus in the Mediterranean Spanish population (sample 1)

| | PLIN1 | | PLIN4 | | PLIN5 | | PLIN6 | |
|---|---|---|---|---|---|---|---|---|
| | Men | Women | Men | Women | Men | Women | Men | Women |
| Genotypes | n (%) | n (%) | n (%) | n (%) | n (%) | n (%) | n (%) | n (%) |
| 11 | 309 (40.8) | 331 (42.4) | 405 (52.5) | 451 (57.7) | 282 (36.2) | 318 (40.5) | 328 (44.6) | 346 (44.7) |
| 12 | 334 (44.1) | 342 (43.8) | 307 (39.8) | 271 (34.7) | 380 (48.7) | 345 (43.9) | 321 (43.7) | 333 (43.0) |
| 22 | 114 (15.1) | 108 (13.8) | 60 (7.8) | 59 (7.6) | 118 (15.1) | 122 (15.5) | 86 (11.7) | 95 (12.3) |

Allele frequency and 95% CI

| | | | | |
|---|---|---|---|---|
| Allele 2 | 0.364 (0.347-0.381) | 0.262 (0.247-0.278) | 0.385 (0.368-0.402) | 0.337 (0.320-0.353) |

Linkage disequilibrium between variants: D; D′ and (p)

| | | | | |
|---|---|---|---|---|
| PLIN1 | — | 0.159; 0.958 (p < 0.001) | 0.033; 0.149 (p < 0.001) | 0.085; 0.394 (p < 0.001) |
| PLIN4 | | | 0.031; 0.191 (p < 0.001) | 0.078; 0.453 (p < 0.001) |
| PLIN5 | | | | 0.066; 0.320 (p < 0.001) |
| PLIN6 | | | | — |

CI: Confidence interval
Differences by gender aoross genotypes were nonsignificant for PLIN1 (p = 0.727), PLIN4 (p = 0.097), PLIN5 (p = 0.142) or PLIN6 (p = 0.932) polymorphisms. Thus, allele frequencies and linkage desequilibrium between polymorphisms has been estimated for men and women.
D: Linkage disequilibrium coefficient
D′: Linkage disequilibrium coefficient D standardized by the maximum value it can take (D/Dmax)

TABLE 4
Frecuency of the 16 detected haplotypes of the
four PLIN loci in sample 1 (men + women)

Haplotypes

| PLIN1 | PLIN4 | PLIN5 | PLIN6 | Frequency |
|---|---|---|---|---|
| 1 | 1 | 1 | 1 | 0.3885 |
| 1 | 1 | 1 | 2 | 0.0368 |
| 1 | 1 | 2 | 1 | 0.1250 |
| 1 | 1 | 2 | 2 | 0.0879 |
| 1 | 2 | 1 | 1 | 0.0046 |
| 1 | 2 | 1 | 2 | 0.0007 |
| 1 | 2 | 2 | 1 | 0.0001 |
| 1 | 2 | 2 | 2 | 0.0026 |
| 2 | 1 | 1 | 1 | 0.0401 |
| 2 | 1 | 1 | 2 | 0.0197 |
| 2 | 1 | 2 | 1 | 0.0184 |
| 2 | 1 | 2 | 2 | 0.0247 |
| 2 | 2 | 1 | 1 | 0.0435 |
| 2 | 2 | 1 | 2 | 0.0809 |
| 2 | 2 | 2 | 1 | 0.0459 |
| 2 | 2 | 2 | 2 | 0.0807 |

TABLE 5
Body mass index (BMI) and obesity-related phenotypes according to the carrier status of the allele 2
variant at each one of the PLIN polymorphisms in the Mediterranean Spanish population (sample 1).
Age-adjusted means in men.

| | MEN | | | | | |
|---|---|---|---|---|---|---|
| | PLIN1 | | | PLIN4 | | |
| | 11 (n = 309) | 12 + 22 (n = 448) | | 11 (n = 405) | 12 + 22 (n = 367) | |
| | Mean (SE) | Mean (SE) | P | Mean (SE) | Mean (SE) | P |
| BMI (kg/m2) | 26.4 (0.2) | 26.4 (0.2) | 0.926 | 26.3 (0.2) | 26.5 (0.2) | 0.776 |
| Weight (Kg) | 78.8 (0.6) | 78.8 (0.5) | 0.959 | 78.6 (0.5) | 78.9 (0.5) | 0.643 |
| Waist-to-hip ratio | 0.95 (0.01) | 0.95 (0.01) | 0.653 | 0.95 (0.01) | 0.96 (0.01) | 0.181 |
| Glucose (mg/dL) | 94.0 (1.3) | 94.3 (1.1) | 0.764 | 94.3 (1.2) | 92.8 (1.2) | 0.412 |
| Total-C (mg/dL) | 207.9 (2.0) | 206.5 (1.7) | 0.604 | 208.8 (1.8) | 204.5 (1.9) | 0.102 |
| LDL-C (mg/dL) | 136.9 (2.0) | 134.4 (1.7) | 0.350 | 137.1 (1.8) | 133.0 (1.9) | 0.122 |
| HDL-C (mg/dL) | 45.7 (0.6) | 46.8 (0.5) | 0.121 | 46.0 (0.5) | 46.8 (0.5) | 0.264 |
| TG (mg/dL) | 130.0 (4.8) | 133.7 (4.5) | 0.459 | 130.1 (4.1) | 134.8 (4.4) | 0.332 |
| SBP (mmHg) | 124.8 (0.8) | 124.7 (0.7) | 0.923 | 124.5 (0.7) | 124.7 (0.8) | 0.867 |
| DBP (mmHg) | 75.5 (0.6) | 75.9 (0.5) | 0.509 | 75.1 (0.5) | 76.2 (0.5) | 0.142 |
| | MEN | | | | | |
| | PLIN5 | | | PLIN6 | | |
| | 11 (n = 282) | 12 + 22 (n = 498) | | 11 (n = 328) | 12 + 22 (n = 407) | |
| | Mean (SE) | Mean (SE) | P | Mean (SE) | Mean (SE) | P |
| BMI (kg/m2) | 26.2 (0.2) | 26.4 (0.1) | 0.396 | 26.4 (0.2) | 26.4 (0.2) | 0.756 |
| Weight (Kg) | 78.3 (0.6) | 78.9 (0.4) | 0.466 | 78.9 (0.6) | 78.8 (0.5) | 0.803 |
| Waist-to-hip ratio | 0.95 (0.01) | 0.95 (0.01) | 0.682 | 0.95 (0.01) | 0.95 (0.01) | 0.961 |
| Glucose (mg/dL) | 94.3 (1.4) | 93.6 (1.1) | 0.659 | 94.4 (1.3) | 94.9 (1.2) | 0.817 |
| Total-C (mg/dL) | 205.0 (2.1) | 207.0 (1.7) | 0.426 | 207.6 (1.9) | 205.7 (1.8) | 0.491 |
| LDL-C (mg/dL) | 133.4 (2.2) | 135.5 (1.7) | 0.434 | 135.2 (2.0) | 134.6 (1.8) | 0.837 |
| HDL-C (mg/dL) | 45.9 (0.6) | 46.8 (0.5) | 0.487 | 45.7 (0.6) | 46.7 (0.5) | 0.192 |
| TG (mg/dL) | 129.2 (4.9) | 133.6 (4.8) | 0.330 | 133.1 (4.7) | 133.9 (4.3) | 0.896 |
| SBP (mmHg) | 123.6 (0.9) | 125.4 (0.7) | 0.108 | 125.3 (0.8) | 124.7 (0.7) | 0.605 |
| DBP (mmHg) | 74.9 (0.6) | 76.0 (0.5) | 0.123 | 75.5 (0.6) | 76.0 (0.5) | 0.498 |

SE: Standard error
Total-C: Total cholesterol.
LDL-C: low-density lipoprotein cholesterol.
HDL-C: high-density lipoprotein-cholesterol,
TG: triglycerides,
SBP: Systolic blood pressure.
DBP:diastolic blood pressure.
Weight was additionally adjusted for height.

TABLE 6
Body mass index (BMI) and obesity-related phenotypes according to the carrier status
of the allele 2 variant at each one of the polymorphisms in the Mediterranean Spanish
population (sample 1). Age-adjusted means in women.

| | WOMEN | | | | | |
|---|---|---|---|---|---|---|
| | PLIN1 | | | PLIN4 | | |
| | | 12 + | | | 12 + | |
| | 11 (n = 331) | 22 (n = 450) | | 11 (n = 451) | 22 (n = 330) | |
| | Mean (SE) | Mean (SE) | P | Mean (SE) | Mean (SE) | P |
| BMI (kg/m2) | 26.3 (0.3) | 25.3 (0.2) | 0.004 | 26.1 (0.2) | 25.2 (0.3) | 0.004 |
| Weight (Kg) | 65.7 (0.6) | 63.5 (0.5) | 0.007 | 65.4 (0.6) | 63.2 (0.6) | 0.011 |
| Waist-to-hip ratio | 0.86 (0.01) | 0.86 (0.01) | 0.519 | 0.87 (0.01) | 0.85 (0.01) | 0.032 |
| Glucose (mg/dL) | 97.8 (0.9) | 95.5 (0.9) | 0.090 | 97.9 (0.8) | 94.5 (1.0) | 0.008 |
| Total-C (mg/dL) | 202.1 (1.8) | 201.1 (1.6) | 0.652 | 201.3 (1.6) | 201.4 (1.8) | 0.962 |
| LDL-C (mg/dL) | 127.9 (1.8) | 128.6 (1.5) | 0.761 | 127.1 (1.5) | 129.9 (1.7) | 0.222 |
| HDL-C (mg/dL) | 54.3 (0.6) | 54.8 (0.5) | 0.498 | 54.2 (0.5) | 55.0 (0.6) | 0.361 |
| TG (mg/dL) | 99.5 (3.0) | 95.1 (2.6) | 0.099 | 102.5 (2.6) | 89.4 (2.9) | 0.005 |
| SBP (mmHg) | 124.2 (0.9) | 122.0 (0.8) | 0.097 | 123.5 (0.8) | 121.9 (0.9) | 0.198 |
| DBP (mmHg) | 75.5 (0.6) | 74.1 (0.5) | 0.105 | 74.8 (0.5) | 74.6 (0.6) | 0.841 |
| | WOMEN | | | | | |
| | PLIN5 | | | PLIN6 | | |
| | | 12 + | | | 12 + | |
| | 11 (n = 318) | 22 (n = 467) | | 11 (n = 346) | 22 (n= 428) | |
| | Mean (SE) | Mean (SE) | P | Mean (SE) | Mean (SE) | P |
| BMI (kg/m2) | 25.8 (0.3) | 25.7 (0.2) | 0.965 | 25.9 (0.4) | 25.7 | 0.456 |
| Weight (Kg) | 64.5 (0.6) | 64.4 (0.5) | 0.844 | 64.9 (0.6) | 64.2 (0.6) | 0.385 |
| Waist-to-hip ratio | 0.86 (0.01) | 0.87 (0.01) | 0.172 | 0.87 (0.01) | 0.86 (0.01) | 0.299 |
| Glucose (mg/dL) | 96.8 (0.9) | 96.6 (0.8) | 0.862 | 96.9 (0.9) | 96.7 (0.9) | 0.908 |
| Total-C (mg/dL) | 201.1 (1.7) | 202.3 (1.6) | 0.645 | 200.8 (1.8) | 202.3 (1.6) | 0.650 |
| LDL-C (mg/dL) | 127.8 (1.8) | 128.9 (1.5) | 0.653 | 127.7 (1.7) | 129.2 (1.6) | 0.442 |
| HDL-C (mg/dL) | 54.1 (0.6) | 54.9 (0.5) | 0.245 | 53.8 (0.6) | 55.1 (0.6) | 0.120 |
| TG (mg/dL) | 102.0 (3.0) | 95.4 (2.6) | 0.207 | 100.1 (2.9) | 95.3 (2.6) | 0.314 |
| SBP (mmHg) | 122.7 (0.9) | 123.7 (0.8) | 0.433 | 123.2 (0.9) | 122.6 (0.8) | 0.624 |
| DBP (mmHg) | 74.4 (0.6) | 75.0 (0.5) | 0.410 | 74.4 (0.6) | 75.9 (0.5) | 0.562 |

SE: Standard error

TABLE 7
Combined effect of the PLIN polymorphisms on weight and BMI in men and women from
sample 1 and sample 2

| | | | | | WOMEN | | | | | MEN | | | | |
|---|---|---|---|---|---|---|---|---|---|---|---|---|---|---|
| | PLIN POLYMORPHISMS | | | | Weight | | | BMI | | Weight | | | BMI | |
| Group | PLIN1 | PLIN4 | PLIN5 | PLIN6 | n | Mean (SE) | P | Mean (SE) | P | n | Mean (SE) | P | Mean (SE) | P |
| | | | | | | | 0.007 | | 0.005 | | | 0.991 | | 0.995 |
| 1 | 11 | 11 | 11 | 11 | 129 | 69.5 (1.5) | 0.047³ | 27.7 (0.6) | 0.043³ | 107 | 81.3 (1.4) | | 27.0 (0.5) | |
| 2 | 11 | 11 | 12 or 22 | 12 or 22 | 108 | 72.2 (1.6) | 0.009³ | 28.7 (0.6) | 0.006³ | 78 | 81.9 (1.7) | | 27.2 (0.5) | |
| 3 | 12 or 22 | 12 or 22 | 11 | 11 | 29 | 62.9 (3.1) | <0.05^{1,2} | 24.8 (1.2) | <0.05^{1,2} | 24 | 80.9 (3.0) | | 26.9 (0.9) | |
| 4 | 12 or 22 | 12 or 22 | 12 or 22 | 12 or 22 | 184 | 66.1 (1.3) | 0.003² | 26.3 (0.5) | 0.003² | 178 | 81.5 (1.1) | | 27.0 (0.4) | |

TABLE 8
Frequencies of PLIN haplotypes according to the obese/non-
obese status and haplotypic ORs estimates in women

| PLIN SNP | | | | Non-obese | Obese | | 95% CI | |
|---|---|---|---|---|---|---|---|---|
| 6209 | 11482 | 13041 | 14995 | (n = 237) | (n = 122) | OR* | Lower | Upper |

4 SNP haplotype^{†}

| | | | | | | | | |
|---|---|---|---|---|---|---|---|---|
| T | G | A | A | 0.306 | 0.267 | 1^{§} | | |
| T | G | G | A | 0.133 | 0.112 | 0.81 | 0.40 | 1.63 |
| T | G | A | T | 0.045 | 0.063 | 1.36 | 0.47 | 3.91 |
| T | G | G | T | 0.039 | 0.072 | 2.09 | 0.83 | 5.23 |
| C | G | A | A | 0.082 | 0.047 | 0.58 | 0.25 | 1.34 |
| C | A | A | A | 0.089 | 0.065 | 0.77 | 0.31 | 1.92 |
| C | A | G | T | 0.067 | 0.103 | 1.79 | 0.82 | 3.92 |
| C | A | A | T | 0.109 | 0.120 | 1.21 | 0.58 | 2.52 |

2 SNP haplotype (13041 and 14995)^{‡}

| | | | | | | |
|---|---|---|---|---|---|---|
| A | A | 0.485 | 0.371 | 1^{§} | | |
| A | T | 0.201 | 0.250 | 1.76 | 1.07 | 2.90 |
| G | A | 0.165 | 0.192 | 1.44 | 0.81 | 2.55 |
| G | T | 0.149 | 0.187 | 1.73 | 1.06 | 2.82 |

*Multiple adjustment for Age, smoking, alcohol consumption, and medication status
^{†}Likelihood ratio test a global haplotype effect: LRT statistic = 11.82, with 7 degrees of freedom (df), P = 0.107
^{‡}Likelihood ratio test a global haplotype effect: LRT statistic = 8.60, with 3 df, P = 0.035
^{§}Haplotype treated as reference

TABLE 9
Plasma Lipid and glucose measures* by PLIN genotypes in women

| | Genotypes | | | P^{†} | Genotypes | | | P^{†} |
|---|---|---|---|---|---|---|---|---|
| | PLIN 6209 T > C | | | | PLIN 11482 G > A | | | |
| | TT (n = 103) | TC (n = 168) | CC (n = 80) | | GG (n = 163) | GA (n = 154) | AA (n = 34) | |
| FG (mg/dL) | 94.2 (2.7) | 97.0 (2.1) | 95.8 (3.1) | 0.831 | 96.3 (2.2) | 95.4 (2.2) | 96.5 (4.7) | 0.998 |
| TG (mg/dL) | 153.3 (7.5) | 155.5 (5.8) | 147.6 (8.5) | 0.914 | 147.9 (5.9) | 159.8 (6.0) | 150.6 (12.9) | 0.186 |
| TC (mg/dL) | 215.3 (3.9) | 210.8 (3.0) | 219.8 (4.4) | 0.223 | 211.5 (3.1) | 214.3 (3.1) | 227.7 (6.7) | 0.090 |
| LDL-C (mg/dL) | 123.7 (3.3) | 115.8 (2.6) | 129.8 (3.7) | 0.006 | 121.1 (2.6) | 118.6 (2.7) | 136.2 (5.7) | 0.021 |
| HDL-C (mg/dL) | 61.0 (1.4) | 63.5 (1.1) | 60.7 (1.6) | 0.190 | 61.0 (1.1) | 63.5 (1.1) | 61.8 (2.3) | 0.265 |
| TC/HDL-C | 3.73 (0.10) | 3.48 (0.08) | 3.71 (0.11) | 0.078 | 3.63 (0.08) | 3.56 (0.08) | 3.69 (0.17) | 0.705 |
| | PLIN 13041 A > G) | | | | PLIN 14995 A > T | | | |
| | AA (n = 151) | AG (n = 164) | GG(n = 36) | | AA (n = 138) | AT (n = 159) | TT (n = 55) | |
| FG (mg/dL) | 93.9 (2.2) | 96.7 (2.2) | 101.2 (4.7) | 0.410 | 93.5 (2.3) | 98.2 (2.2) | 95.2 (3.7) | 0.487 |
| TG (mg/dL) | 147.7 (6.1) | 154.5 (5.9) | 170.2 (12.8) | 0.172 | 145.4 (6.4) | 156.3 (6.0) | 164.0 (10.1) | 0.155 |
| TC (mg/dL) | 210.7 (3.2) | 214.5 (3.0) | 227.2 (6.6) | 0.081 | 212.4 (3.3) | 214.7 (3.1) | 216.7 (5.3) | 0.756 |
| LDL-C (mg/dL) | 120.4 (2.7) | 120.4 (2.6) | 129.4 (5.8) | 0.342 | 120.2 (2.9) | 121.3 (2.7) | 123.7 (4.6) | 0.814 |
| HDL-C (mg/dL) | 61.0 (1.1) | 62.6 (1.1) | 64.8 (2.4) | 0.298 | 63.1 (1.2) | 61.8 (1.1) | 60.7 (1.9) | 0.504 |
| TC/HDL-C | 3.60 (0.08) | 3.57 (0.08) | 3.84 (0.17) | 0.333 | 3.56 (0.08) | 3.65 (0.08) | 3.61 (0.13) | 0.742 |

TC: Total cholesterol.
LDL-C: low-density lipoprotein cholesterol.
HDL-C: high-density lipoprotein-cholesterol,
TG: triglycerides;
FG: fasting glucose.
*Presented as mean (standard error).
^{†}Test of homogeneity, with multiple adjustment for age, BMI, tobacco smoking, alcohol consumption, and medication status.

TABLE 10
Plasma Lipid and glucose measures* by PLIN genotypes in men

| | Genotypes | | | P^{†} | Genotypes | | |
|---|---|---|---|---|---|---|---|
| | PLIN 6209 T > C | | | | PLIN 11482 G > A | | |
| | TT (n = 118) | TC (n = 162) | CC (n = 75) | | GG (n = 189) | GA (n = 131) | AA (n = 34) |
| FG (mg/dL) | 107.4 (3.2) | 106.8 (2.7) | 107.9 (4.1) | 0.992 | 109.4 (2.6) | 105.3 (3.1) | 103.2 (6.0) |
| TG (mg/dL) | 186.6 (10.1) | 189.9 (8.6) | 192.5 (12.9) | 0.791 | 190.3 (8.0) | 189.2 (9.6) | 182.0 (19.0) |
| TC (mg/dL) | 211.1 (4.1) | 206.4 (3.5) | 208.7 (5.2) | 0.675 | 206.7 (3.2) | 210.3 (3.9) | 212.1 (7.6) |
| LDL-C (mg/dL) | 124.6 (3.3) | 123.5 (2.8) | 125.1 (4.2) | 0.938 | 121.0 (2.6) | 128.1 (3.2) | 127.2 (6.1) |
| HDL-C (mg/dL) | 48.0 (1.1) | 47.3 (0.9) | 46.2 (1.4) | 0.585 | 47.9 (0.9) | 46.0 (1.1) | 49.2 (2.1) |
| TC/HDL-C | 4.65 (0.12) | 4.57 (0.11) | 4.77 (0.16) | 0.582 | 4.56 (0.10) | 4.74 (0.12) | 4.61 (0.23) |
| | PLIN 13041 A > G | | | | PLIN 14995 A > T | | |
| | AA (n = 160) | AG (n = 151) | GG(n = 44) | | AA (n = 158) | AT (n = 151) | TT (n = 44) |
| FG (mg/dL) | 110.0 (2.8) | 104.6 (2.8) | 105.8 (5.4) | 0.476 | 109.9 (2.8) | 104.8 (2.9) | 106.3 (5.4) |
| TG (mg/dL) | 191.3 (8.6) | 196.4 (8.9) | 157.8 (16.7) | 0.153 | 197.6 (8.8) | 183.4 (9.0) | 180.8 (16.7) |
| TC (mg/dL) | 214.7 (3.5) | 203.1 (3.6) | 203.8 (6.7) | 0.051 | 208.7 (3.5) | 207.0 (3.6) | 213.8 (6.7) |
| LDL-C (mg/dL) | 129.3 (2.8) | 119.7 (2.9) | 120.6 (5.4) | 0.049 | 122.6 (2.9) | 124.7 (2.9) | 128.7 (5.4) |
| HDL-C (mg/dL) | 47.6 (0.9) | 45.9 (1.0) | 50.9 (1.8) | 0.047 | 46.7 (1.0) | 47.5 (1.0) | 49.3 (1.8) |
| TC/HDL-C | 4.75 (0.11) | 4.62 (0.11) | 4.30 (0.21) | 0.158 | 4.71 (0.11) | 4.60 (0.11) | 4.52 (0.21) |

TC: Total cholesterol.
LDL-C: low-density lipoprotein cholesterol.
HDL-C: high-density lipoprotein-cholesterol,
TG: triglycerides;
FG: fasting glucose.
*Presented as mean (standard error).
^{†}Test of homogeneity, with multiple adjustment for age, BMI, tobacco smoking, alcohol consumption, and medication status.

TABLE 11
Descriptive characteristics¹ of Singapore population by gender and ethnics

| | Singapore | | | | | |
|---|---|---|---|---|---|---|
| | Chinese | | Malay | | Indian | |
| | Men | Women | Men | Women | Men | Women |
| | (n = 1263) | (n = 1500) | (n = 360) | (n = 386) | (n = 286) | (n = 312) |
| Age (years) | 38.2 ± 12.3 | 37.8 ± 12.2 | 39.6 ± 12.7 | 38.4 ± 12.7 | 41.3 ± 12.1 | 40.0 ± 12.1 |
| BMI (kg/m²) | 23.5 ± 3.7 | 22.1 ± 3.6 | 24.7 ± 4.0 | 26.3 ± 5.6 | 24.6 ± 4.0 | 25.6 ± 5.0 |
| Total-C (mmol/l) | 5.52 ± 1.04 | 5.33 ± 1.05 | 5.88 ± 1.13 | 5.73 ± 1.17 | 5.72 ± 1.17 | 5.33 ± 1.03 |
| LDL-C (mmol/l) | 3.54 ± 0.95 | 3.24 ± 0.93 | 3.95 ± 1.02 | 3.75 ± 1.13 | 3.88 ± 1.08 | 3.53 ± 0.96 |
| HDL-C (mmol/l) | 1.27 ± 0.32 | 1.56 ± 0.37 | 1.15 ± 0.28 | 1.44 ± 0.33 | 1.06 ± 0.29 | 1.23 ± 0.31 |
| Fasting TG (mmol/l) | 1.69 ± 1.55 | 1.16 ± 0.75 | 2.00 ± 1.59 | 1.39 ± 0.88 | 2.08 ± 1.78 | 1.33 ± 0.68 |
| Obesity (%)² | 54(4.28) | 46(3.07) | 29(8.06) | 94(24.35) | 22(7.69) | 55(17.63) |
| Overweight (%)² | 401(15.36) | 140(9.33) | 93(25.83) | 152(39.38) | 70(24.48) | 117(37.50) |
| Current smoker (%) | 298(23.36) | 45(3.00) | 162(45.00) | 15(3.89) | 87(30.42) | 1(0.32) |
| Alcohol user (%) | 749(59.30) | 494(32.93) | 44(12.22) | 12(3.11) | 149(52.10) | 55(17.63) |
| Diabetes milletus (%) | 40(3.17) | 24(1.60) | 16(4.44) | 21(5.44) | 27(9.44) | 24(7.69) |

¹Continuous variables were presented as mean ± SD, while categorical variables were presented as the number of cases and percentages of prevalence.
²Obesity: BMI >= kg/m2; Overweight: BMI >= 25 kg/m²
Total-C: Total cholesterol.
LDL-C: low-density lipoprotein cholesterol.
HDL-C: high-density lipoprotein cholesterol,
TG: triglycerides.

TABLE 12
PLIN haplotype frequency in obese subjects and non-obese control in Chinese and OR estimates

| Inferred haplotype | | | | | | Non-obese | Obese | | | | |
|---|---|---|---|---|---|---|---|---|---|---|---|
| Code³ | 6209 | 10171 | 11482 | 13041 | 14995 | (n = 2663) | (n = 100) | OR (95% CI) | P | Adj-OR (95% CI)¹ | P |

5 SNP haplotype

| | | | | | | | | | | | |
|---|---|---|---|---|---|---|---|---|---|---|---|
| 21111 | T | A | G | A | A | 0.220 | 0.256 | 1² | | 1² | |
| 11222 | C | A | A | G | T | 0.173 | 0.178 | 1.05 (0.67-1.63) | 0.8387 | 1.02 (0.65-1.60) | 0.9298 |
| 11212 | C | A | A | A | T | 0.191 | 0.215 | 1.14 (0.76-1.70) | 0.5190 | 1.20 (0.79-1.82) | 0.3892 |
| 12111 | C | T | G | A | A | 0.189 | 0.183 | 0.99 (0.62-1.58) | 0.9625 | 1.04 (0.65-1.69) | 0.8586 |
| 21121 | T | A | G | G | A | 0.052 | 0.044 | 0.85 (0.32-2.22) | 0.7375 | 1.17 (0.59-2.29) | 0.6544 |
| 12121 | C | T | G | G | A | 0.041 | 0.034 | 0.83 (0.30-2.28) | 0.7204 | 0.83 (0.31-2.24) | 0.7100 |

3 SNP haplotype

| | | | | | | | | | |
|---|---|---|---|---|---|---|---|---|---|
| 111 | G | A | A | 0.414 | 0.270 | 1² | | 1² | |
| 212 | A | A | T | 0.192 | 0.249 | 1.07 (0.74-1.56) | 0.7154 | 1.10 (0.75-1.63) | 0.6230 |
| 222 | A | G | T | 0.174 | 0.227 | 0.98 (0.65-1.49) | 0.9351 | 0.95 (0.62-1.46) | 0.8251 |
| 121 | G | G | A | 0.108 | 0.129 | 0.76 (0.37-1.58) | 0.4619 | 0.75 (0.34-1.62) | 0.4588 |
| 211 | A | G | T | 0.036 | 0.043 | 0.88 (0.33-2.37) | 0.8064 | 0.83 (0.31-2.22) | 0.7097 |
| 112 | G | A | T | 0.035 | 0.048 | 0.76 (0.37-1.56) | 0.4562 | 0.82 (0.38-1.74) | 0.6006 |

¹Adjusted for age, sex, smoking, alcohol consumption, exercise, and diabetes status
²Used as reference haplotype
³1 represent the common allele, and, 2 represent the minor allele

TABLE 13
PLIN haplotype frequency in obese subjects and non-obese control in Malays and OR estimates

| Inferred haplotype | | | | | | Non-obese | Obese | | | | |
|---|---|---|---|---|---|---|---|---|---|---|---|
| Code³ | 6209 | 10171 | 11482 | 13041 | 14995 | (n = 2663) | (n = 100) | OR (95% CI) | P | Adj-OR (95% CI)¹ | P |

5 SNP haplotype

| | | | | | | | | | | | |
|---|---|---|---|---|---|---|---|---|---|---|---|
| 21111 | T | A | G | A | A | 0.241 | 0.159 | 1² | | 1² | |
| 11222 | C | A | A | G | T | 0.173 | 0.229 | 1.64 (1.08-2.48) | 0.0197 | 1.67 (1.07-2.60) | 0.0227 |
| 11212 | C | A | A | A | T | 0.189 | 0.248 | 1.65 (1.11-2.46) | 0.0141 | 1.55 (1.01-2.38) | 0.0437 |
| 12111 | C | T | G | A | A | 0.153 | 0.102 | 0.80 (0.44-1.44) | 0.4536 | 0.82 (0.45-1.50) | 0.5316 |
| 21121 | T | A | G | G | A | 0.074 | 0.081 | 1.33 (0.71-2.50) | 0.3728 | 1.17(0.59-2.29) | 0.6544 |
| 11211 | C | A | A | G | T | 0.034 | 0.037 | 1.30 (0.54-3.11) | 0.5561 | 1.23(0.52-2.93) | 0.6389 |

3 SNP haplotype

| | | | | | | | | | |
|---|---|---|---|---|---|---|---|---|---|
| 111 | G | A | A | 0.414 | 0.270 | 1² | | 1² | |
| 212 | A | A | T | 0.192 | 0.249 | 2.12 (1.36-3.32) | 0.0010 | 2.04 (1.28-3.25) | 0.0029 |
| 222 | A | G | T | 0.174 | 0.227 | 2.02 (1.36-3.01) | 0.0005 | 2.05 (1.35-3.12) | 0.0007 |
| 121 | G | G | A | 0.108 | 0.129 | 1.89 (1.05-3.41) | 0.0332 | 1.59 (0.87-2.90) | 0.1331 |
| 211 | A | G | T | 0.036 | 0.043 | 1.84 (0.71-4.78) | 0.2120 | 1.81 (0.70-4.67) | 0.2213 |
| 112 | G | A | T | 0.035 | 0.048 | 2.30 (0.97-5.30) | 0.0599 | 2.25 (0.96-5.25) | 0.0610 |

¹Adjusted for age, sex, smoking, alcohol consumption, exercise, and diabetes status
²Used as reference haplotype
³1 represent the common allele, and, 2 represent the minor allele

TABLE 14
PLIN haplotype frequency in obese subjects and non-obese control in Indians and OR estimates

| Inferred haplotype | | | | | | Non-obese | Obese | | | | |
|---|---|---|---|---|---|---|---|---|---|---|---|
| Code | 6209 | 10171 | 11482 | 13041 | 14995 | (n = 521) | (n = 77) | OR (95% CI) | P | Adj-OR (95% CI)¹ | P |

5 SNP haplotype

| | | | | | | | | | | | |
|---|---|---|---|---|---|---|---|---|---|---|---|
| 21111 | T | A | G | A | A | 0.247 | 0.237 | 1² | | 1² | |
| 11222 | C | A | A | G | T | 0.179 | 0.154 | 0.87 (0.53-1.42) | 0.5708 | 0.80 (0.46-1.37) | 0.4186 |
| 11212 | C | A | A | A | T | 0.078 | 0.154 | 1.94 (1.06-3.53) | 0.0305 | 1.67 (0.87-3.22) | 0.1234 |
| 12111 | C | T | G | A | A | 0.082 | 0.025 | 0.30 (0.09-1.06) | 0.0606 | 0.29 (0.08-1.07) | 0.0624 |
| 21121 | T | A | G | G | A | 0.157 | 0.160 | 0.97 (0.54-1.76) | 0.9176 | 0.91 (0.49-1.70) | 0.7722 |
| 12121 | C | T | G | G | A | 0.051 | 0.052 | 1.04 (0.44-2.46) | 0.9221 | 0.965 (0.39-2.40) | 0.9387 |

3 SNP haplotype

| | | | | | | | | | |
|---|---|---|---|---|---|---|---|---|---|
| 111 | G | A | A | 0.363 | 0.304 | 1² | | 1² | |
| 212 | A | A | T | 0.087 | 0.161 | 2.39 (1.26-4.50) | 0.0073 | 2.16 (1.10-4.26) | 0.0261 |
| 222 | A | G | T | 0.181 | 0.152 | 0.98 (0.58-1.66) | 0.9368 | 0.90 (0.51-1.59) | 0.7158 |
| 121 | G | G | A | 0.232 | 0.224 | 1.16 (0.70-1.95) | 0.5577 | 1.11 (0.63-1.95) | 0.7147 |
| 211 | A | G | T | 0.043 | 0.034 | 0.77 (0.19-3.17) | 0.7141 | 0.71 (0.15-3.40) | 0.6656 |
| 122 | G | G | T | 0.049 | 0.075 | 2.03 (0.94-4.39) | 0.0714 | 2.08 (0.93-4.67) | 0.0751 |

¹Adjusted for age, sex, smoking, alcohol consumption, exercise, and diabetes status
²Used as reference haplotype
³1 represent the common allele, and, 2 represent the minor allele

TABLE 15
OBESITY PROTECTIVE

| | Haplotypes with | | | | | | | | | |
|---|---|---|---|---|---|---|---|---|---|---|
| | Decreased | | | | | | | | | |
| | Risk of Obesity | | | | | | | | | |
| LOCUS | (Caucasian) | | (Mediterranian) | | (Malay) | | | (Indian) | | |
| | a | b | c | d | E | f | g | h | i | j |
| PLIN1 | | C | C | C | T | C | C | C | C | C |
| PLIN3 | | | | | T | A | T | A | A | T |
| PLIN4 | | G | A | A | G | G | | G | G | |
| PLIN5 | A | A | | A | A | A | | A | A | |
| PLIN6 | A | A | | A | A | A | | A | A | |

TABLE 16
DIAGNOSIS FOR INCREASED RISK OF OBESITY

| | Halotypes with | | | | | | | | | | | | | | | |
|---|---|---|---|---|---|---|---|---|---|---|---|---|---|---|---|---|
| | | | | Increased | | | | | | | | | | | | |
| | | | | Risk of Obesity | | | | | | | | | | | | |
| | (Caucasian) | | | (Mediterranian) | | | (Malay) | | | | | | (Indian) | | | |
| LOCUS | k | l | M | n | o | p | Q | r | s | t | u | v | w | x | y | z |
| PLIN1 | | | T | T | T | T | T | T | | | | T | T | | | T |
| PLIN3 | | | | | | | A | A | | | | A | A | | | A |
| PLIN4 | | | G | G | G | G | A | A | A | A | G | | A | A | G | |
| PLIN5 | G | A | G | | A | G | A | G | A | G | G | | A | A | G | |
| PLIN6 | T | T | T | | A | T | T | T | T | T | A | | T | T | T | |

1-21. (canceled)

22. A method of determining an increased risk of obesity and obesity-related diseases in an individual comprising the steps of:
a) Genotyping the PLIN1 6209T/C, PLIN3 10171A/T, PLIN4 11482G/A, PLIN5 13041A/G, PLIN6 14995 A/T loci from a biological sample taken from the individual;
b) Creating a haplotype based on the PLIN genotypes as determined in step (a);
c) and wherein a haplotype selected from the group consisting of PLIN5-G/PLIN6T; PLIN5-A/PLIN6-T; PLIN1-T/PLIN4-G/PLIN5-G/PLIN6-T; PLIN1-T/PLIN4-G; PLIN1-T/PLIN4-G/PLIN5-A/PLIN6-A; PLIN1-T/PLIN3-A/PLIN4-A/PLIN5-A/PLIN6-T; PLIN1-T/PLIN3-A/PLIN4-A/PLIN5-G/PLIN6-T; PLIN4-A/PLIN5-A/PLIN6T; PLIN4-A/PLIN5-G/PLIN6-T; PLIN4-G/PLIN5-G/PLIN6-A; PLIN1-T/PLIN3-A; is indicative of increased risk of obesity and obesity-related diseases in the individual.

23. The method of claim 22, further comprising a step of determining the ethnic background of the individual, wherein,
a) when the individual is of Caucasian descent and the haplotype is PLIN5-G/PLIN6-T; PLIN5-A/PLIN6-T; or PLIN 1-T/PLIN4-G/PLIN5-G/PLIN6-T the individual has an increased risk of obesity and obesity-related diseases;
b) when the individual is of Mediterranean descent and the haplotype is PLIN1-T/PLIN4-G; PLIN1-T/PLIN4-G/PLIN5-A/PLIN6-A; or PLIN1-T/PLIN4-G/PLIN5-G/PLIN6-T the individual has an increased risk of obesity and obesity-related diseases;
c) when the individual is of Malayan descent and the haplotype is PLIN1/T/PLIN3-A/PLIN 4-A/PLIN5-A/PLIN6-T; PLIN 1-T/PLIN3-A/PLIN 4-A/PLIN5-G/PLIN6-T; PLIN4-A/PLIN 5-A/PLIN6-T; PLIN4-A/PLIN5-G/PLIN6-T; PLIN4G/PLIN5G/PLIN6-A; or PLIN 1-T/PLIN3-A the individual has an increased risk of obesity and obesity-related; and
d) when the individual is of Indian descent and the haplotype is PLIN1-T/PLIN3-A/PLIN4-A/PLIN5-A/PLIN6-T; PLIN4-APLIN5-A/PLIN6T; PLIN4-G/PLIN5-G/PLIN6-T; or PLIN1-T/PLIN3-A the individual has an increased risk of obesity and obesity-related diseases.

24. The method as claimed in claim 22 wherein the individual is of Caucasian descent, comprising genotyping the PLIN5 13041A/G and PLIN6 14995 A/T loci from the biological sample taken from the individual, wherein homozygosity of allele G in the PLIN5 locus or homozygosity of allele T in the PLIN 6 locus is indicative of increased risk of obesity and obesity-related diseases in the individual of Caucasian descent.

25. The method as claimed in claim 22 wherein the individual is of Malayan or Indian descent, comprising genotyping the PLIN6 14995 A/T loci from the biological sample taken from the individual, wherein homozygosity of allele T in the PLIN 6 locus is indicative of increased risk of obesity and obesity-related diseases in the individual of Malayan or Indian descent.

26. The method as claimed in claim 22 wherein the individual is of Malayan or Indian descent, comprising genotyping the PLIN4 11482 G/A loci from the biological sample taken from the individual, wherein homozygosity of allele A in the PLIN4 locus in indicative of increased risk of obesity and obesity-related diseases in the individual of Malayan or Indian descent.

27. The method as claimed in claim 22 wherein the individual is of Malayan or Indian descent, comprising genotyping the PLIN5 13041 A/G loci from the biological sample taken from the individual, wherein homozygosity of allele G in the PLIN5 locus in indicative of increased risk of obesity and obesity-related diseases in the individual of Malayan or Indian descent.

28. The method of claim 22, wherein the individual is a woman.

29. The method of claim 22, wherein the individual has been subject to weight reducing diet.

30. The method of claims 22, wherein the obesity-related disease is cardiovascular disease.

31. The method of claim 22, wherein the obesity-related disease is metabolic syndrome.

32. The method of determining a decreased risk of obesity and obesity-related diseases in an individual comprising the steps of:
a) genotyping the PLIN1 6209T/C, PLIN3 10171A/T, PLIN4 11482G/A, PLIN5 13041A/G, PLIN6 14995 A/T loci from a biological sample taken from an individual.
b) creating a haplotype based on the PLIN genotypes as determined in step (a); and
c) wherein a haplotype selected from the group of consisting of PLIN5-A, PLIN6-A; PLIN1-CPLIN4-G/PLIN5-A/PLIN6-A; PLIN1-C/PLIN4-A; PLIN1-C/PLIN4-A/PLIN5-A/PLIN6-A; PLIN1-T/PLIN3-T/PLIN4-G/PLIN5-A/PLIN6-A; PLIN1-C/PLIN3-A/PLIN4-G/PLIN5-A/PLIN6-A; and PLIN1-C/PLIN3-T correlated to the ethnic background of the individual is indicative of decreased risk of obesity and obesity-related disease.

33. The method of claim 32, further comprising the steps of determining the ethnic background of the individual wherein,
a) when the individual is of Caucasian descent and the haplotype is PLIN5-A/PLIN6-A and PLIN1-C/PLIN4-G/PLIN5-A/PLIN6A the individual has a decreased risk of obesity and obesity-related disease.
b) when the individual is of Mediterranean descent and the haplotype is PLIN1-C/PLIN4-A and PLIN1-C/PLIN4A/PLIN5-A/PLIN6-A the individual has a decreased risk of obesity and obesity-related diseases;
c) when the individual is of Malayan descent and the haplotype is PLIN1-T/PLIN3-T/PLIN4-G/PLIN5-A/PLIN6-A; PLIN1-C/PLIN3-A/PLIN4-G/PLIN5-A/PLIN6-A and PLIN1-C/PLIN3-T the individual has a decreased risk of obesity and obesity-related;
d) when the individual is of Indian descent and the haplotype is PLIN1-C/PLIN3-A/PLIN4-G/PLIN5-A/PLIN6-A; PLIN1-C/PLIN3-A/PLIN4-G/PLIN5-A/PLIN6-A; and PLIN1-C/PLIN3-C the individual has a decreased risk of obesity and obesity-related diseases.

34. The method of any of claims 32, wherein the individual is a woman.

35. A kit comprising primer pairs to amplify nucleic acid regions covering PLIN1 6209T/C, PLIN3 10171A/T, PLIN4 11482G/A, PLIN5 13041A/G, and PLIN6 14995 A/T polymorphisms and instructions including the haplotypes associated with increased or decreased risk of obesity and their correlation with an ethnic group.

36. A kit comprising primer pairs of SEQ ID NO: 1 and SEQ ID NO: 2 to amplify nucleic acid region covering PLIN1 polymorphism: SEQ ID NO: 7 and SEQ ID NO: 8 to amplify nucleic acid region covering PLIN3 polymorphism: SEQ ID NO: 10 and SEQ ID NO: 11 to amplify nucleic acid region covering PLIN4 polymorphism; SEQ ID NO: 13 and SEQ ID NO: 14 to amplify nucleic acid region covering PLIN5 polymorphisms; and SEQ ID NO: 16 and SEQ ID NO: 17 to amplify nucleic acid region covering PLIN6 polymorphisms, and instructions including haplotypes associated with increased or decreased risk of obesity and their correlation with an ethnic group.
